SEQUENCE LISTING (2 sequences)

SEQ ID NO: 1 - DNA, 885 bp, Artificial sequence
  Synthesized
atgtgtggtggtagcggtggttcaggtggttctggcggtagtggtggcagcatggttagcaaaggtgaagaggataatatggccatcatcaaagaattcatgcgcttcaaagttcacatggaaggtagcgttaatggccacgaatttgaaattgaaggtgaaggcgaaggtcgtccgtatgaaggcacccagaccgcaaaactgaaagttaccaaaggtggtccgctgccgtttgcatgggatattctgagtccgcagtttatgtatggtagcaaagcctatgttaaacatccggcagatattccggattacctgaaactgagctttccggaaggttttaaatgggaacgtgtgatgaattttgaagatggtggtgttgttaccgttacacaggatagcagcctgcaggatggtgaatttatctataaagttaaactgcgtggcacgaattttccgagtgatggtccggttatgcagaaaaaaaccatgggttgggaagcaagcagcgaacgtatgtatccggaagatggcgcactgaaaggtgaaattaaacagcgtctgaagctgaaagatggcggtcattatgatgcagaagttaaaaccacctacaaagccaaaaaaccggttcagctgcctggtgcatataacgttaacatcaaactggatattaccagccacaacgaggattataccattgtggaacagtatgaacgtgcagaaggtcgccatagtaccggtggtatggatgaactgtataaaggtggcagtggtggatctggtggctcaggcggaagcggtggtagcctgccggaaaccggtggtctgaatgatatttttgaagcccagaaaatcgaatggcatgaacatcatcaccatcaccactaa

SEQ ID NO: 2 - DNA, 80 bp, Artificial sequence
  Synthesized
acgctcggatgccactacaggttggggtcgggcatgcgtccggagaagggcaaacgagaggtcaccagcacgtccatgag

(19) United States
(12) Patent Application Publication
(10) Pub. No.: US 2021/0128735 A1
(43) Pub. Date: May 6, 2021

(54) BIOCOMPATIBLE COPOLYMER CONTAINING MULTIPLE ACTIVE AGENT MOLECULES

(71) Applicant: CIS PHARMA AG, Bubendorf (CH)

(72) Inventors: Christian Geraths, Rheinfelden (DE); Christophe Thommen, Muespach-Le-Haut (FR); Michael Hackebeil, Görwihl (DE); Davide Panighetti, Münchenstein (CH); Hans Hitz, Arisdorf (CH)

(21) Appl. No.: 17/054,050

(22) PCT Filed: May 8, 2019
(86) PCT No.: PCT/EP2019/061769

§ 371 Date: Nov. 9, 2020

Related U.S. Application Data
(60) Provisional application No. 62/762,549, filed on May 10, 2018.

(51) Int. Cl. A61K 47/58; C08F 2/38; A61K 45/06

(52) U.S. Cl. CPC A61K 47/58; C08F 2438/03; A61K 45/06; C08F 2/38

(57) ABSTRACT

The present disclosure relates to the delivery of active agents, e.g., drug substances, using as carriers for their delivery biocompatible copolymers comprising side chain-linked amino acids having active agents bound to their alpha-amino and/or alpha-carboxyl groups, either directly or via linker molecules. The active agent-containing copolymers can be functionalized to contain cell type- or tissue type-specific targeting moieties.

### CROSS-REFERENCE TO RELATED APPLICATIONS

This application claims the benefit of U.S. Provisional Application No. 62/762,549, filed 10 May 2018, which application is incorporated herein by reference in its entirety.

### REFERENCE TO THE SEQUENCE LISTING

The present application is being filed along with a Sequence Listing in electronic format. The Sequence Listing is provided as a file entitled “CIS-010 PCT_ST25.txt”, created 6 May 2019, which is 2 KB in size. The information in the electronic format of the Sequence Listing is incorporated herein by reference in its entirety.

### FIELD OF THE INVENTION

The invention relates to the delivery of active agents, e.g., drug substances, using as carriers for their delivery biocompatible copolymers comprising side chain-linked amino acids having active agents bound to their alpha-amino and/or alpha-carboxyl groups, either directly or via linker molecules.

### BACKGROUND

Cancer is one of the major threats to human health and given the fact that its likelihood is a function of age, the case numbers will increase with ageing of populations. Berger, N A et al. (2006) Cancer in the Elderly, Transactions of the American Clinical and Climatological Association 117: 147-156; Yancik, R (2005) Cancer J. 11: 437-41. In recent years, tumor therapies have made enormous improvements due to the use of tumor-specific agents such as monoclonal antibodies. These antibodies block proliferation signals such as the epidermal growth factor pathway (EGFR) (Cetuximab Erbitux®, Merck KGaA; /Panitumumab, Vectibix®, Amgen/Trastuzumab, Herceptin, Roche) or prevent the formation of new blood vessels by targeting the vascular endothelial growth factor (VEGF) pathway (Bevacizumab, Avastin, Roche) to slow down tumor growth. Since their target antigens are usually over-expressed in tumor tissues, healthy cells are less impaired, wherefore antibody therapies have less off-target effects compared to conventional cytotoxic agents. Zhou, Q. (2017) Biomedicines 5(4); Reichert, J M (2017) MAbs 9: 167-181. The unique specificity of antibodies was also used for combination approaches aimed at targeting cytotoxic drugs to tumor cells. These so-called antibody drug conjugates (ADCs) have proven to be superior to monotherapies with antibodies or cytotoxic agents. Although known since the 1960s, the ADC concept finally encountered interest in the pharmaceutical industry in the clinical development recently, and more than 60 ADCs are undergoing clinical trials. Mullard, A (2013) Nat Rev Drug Discov 12: 329; Beck, A et al. (2017) Nat Rev Drug Discov 16: 315-337.

The first generation of ADCs used free amino groups in the antibodies to attach cytotoxic drugs and drug linker constructs. With up to 80 free amino-groups per antibody, their functionalization leads to highly heterogeneous ADC species with different drug to antibody ratios (DAR) and affinities due to unintended attachment of cytotoxic drugs to the binding interface of the antibodies. The heterogeneity with respect to DARs could be restricted to a certain extent by adjusting the stoichiometry of drug and antibody used in the reaction. With respect to site specificity, the heterogeneity was limited by the chemical accessibility of 1980s when the first clinical trials were conducted. It took another 20 years for the FDA approval of the first ADC. The development of ADCs has dramatically increased since: 30 ADCs have been entering reactive groups. These heterogeneities were also the major issues and regulatory concerns with the first ADCs. Yao, H et al. (2016) Int J Mol Sci 17(2): 194. In addition, the first ADCs were based-upon mouse immunoglobulins known to elicit important immune responses. Because of these drawbacks the first ADC generation failed to show much improvement over conventional therapies, so that gemtuzumab ozogamicin (Mylotarg®), the first FDA-approved ADC, was voluntarily withdrawn from the market by Pfizer in 2010. Beck, A et al. (2017) Nat Rev Drug Discov 16(5): 315-337; Beck, A et al. (2010) Discov Med 10(53): 329-39.

The second generation of ADCs mitigates these difficulties by targeting free thiol groups of the humanized antibodies. These free thiol groups were generated prior to the coupling reaction by mild reduction (e.g., with 1,4-dithiothreitol (DTT)) of 4 inter-chain disulfide bridges in the hinge region of the antibodies. With this strategy, the potential attachment sites could be reduced to 8, resulting in a higher homogeneity of the ADCs. Given the fact that the inter-chain disulfide bonds play a crucial role in antibody integrity, the higher homogeneity was often paid for by negative effects on antibody stability. Although more specific linkers preserving disulfide bridge integrity were designed (as elaborated, e.g., in Shaunak, S et al. (2006) Nat Chem Biol 2(6): 312-3 and Balan, S et al. (2007) Bioconug Chem 18(1): 61-76), ADCs generated suffered from low DARs that were typically about 3-4. If the drug load was further increased, the stability of the antibodies was negatively affected, leading to fast clearance from the blood stream. In addition, the affinity of the antibodies for their tumor cell-specific targets was negatively affected. Beck et al. (2017) Nat Rev Drug Discov 16(5): 315-337; Yao et al. (2016) Int J Mol Sci 17(2): 194; Beck et al. (2010) Discov Med 10(53): 329-39. Since only a few cytotoxic entities were coupled to these antibodies, conventional cytotoxic agents like doxorubicin proved to be insufficiently effective in killing tumor cells. Tolcher, A W (1999) J Clin Oncol 17(2): 478-478. Therefore, novel classes of cytotoxic agents had to be used whose cytotoxicity was several orders of magnitudes higher. Examples for these substances are microtubule inhibitors like mertansine (DM1) or monomethylauristatin E (MMAE). Beck et al. (2017). With such potent drug substances, it is crucial that the toxic payload of ADCs is only released at its target site. Otherwise, severe side effects are likely to result. The linker between the drug and the antibody plays thereby a major role. Recently marketed ADCs like Trastuzumab emtansine (Kadcyla®, Roche) and Brentuximab Vedotin (Adcetris®, Tekada Pharmaceutical) as well as the Mersana concept (Mersana Therapeutics Inc. (Cambridge, Mass.)) use maleimide-based linkers which are known to react with cysteine-bearing proteins, in particular serum albumin. Alley, S C et al. (2008) Bioconjug Chem 19(3): 759-765. Shen, B Q et al. (2012) Nat Biotechnol 30(2): 184-9.

The so-called third generation of ADCs made use of site-specific coupling of the drug to the antibody. A prominent example is Vadatuximab tailirine from Seattle Genetics against acute myeloid leukemia (AML). The ADC contains a genetically engineered cysteine at position 239 in both heavy chains which is used for coupling of pyrrolobenzodiazepine (PBD) dimer that is capable of crosslinking DNA, thereby blocking cell division and causing cell death. The ADC has been successfully tested in a phase I study and is currently in a phase III clinical trial. Beck et al. (2017); Kennedy, D A et al. (2015) Cancer Res 75(15 Supp.), Abstract DDT02-04. Other examples of site-specific coupling of drug to antibody make use of smart tags such as an “aldehyde-tag” (Redwood Biosciences, Catalent) or a “sortase tag” (SMAC-Technology™, NBE Therapeutics; Stefan, N et al. (2017) Mol Cancer Ther 16(5): 879-892). The latter two approaches introduce genetically engineered peptide tags in the antibody to function as specific motives for enzymatic coupling reactions. Third generation ADCs represent more homogeneous products with increased stability but still deliver only a few toxic entities per antibody.

To avoid this limitation, a novel approach that uses a polymeric carrier was recently developed by Mersana Therapeutics. This concept is based on the functionalization of a degradable carrier polymer (referred to as “Fleximers”) with several cytotoxic drug molecules. The drug-loaded polymer is subsequently coupled to a monoclonal antibody by conventional linker chemistry. With this, the DAR could be increased to 12-15 drug molecules per antibody molecule, which drug molecules were distributed over 3-5 attached polymer carriers. “Non-clinical pharmacokinetics of XMT-1522, a HER2 targeting auristatin-based antibody drug conjugate”; poster presentation at the American Association for Cancer Research (AACR) annual meeting in Washington D.C., 2017. Although this approach has many advantages, the resulting ADC contain Fleximer polymers of variable chain length and drug load. In combination with the thiol-maleinimide linker chemistry that was used, the molecular weight of the ADCs vary to some extent. Further, the Fleximer polymer comprises biodegradable ester linkages, raising the issues of long-term storage and/or serum stability Koitka, M et al. (2010) J Pharm Biomed Anal 51(3): 664-78; Li, B et al. (2005) Biochem Pharmacol 70(11: 1673-84.

In addition to antibodies, other target-specific agents including aptamers have been elaborated for blocking or activating aberrant pathways to treat metabolic diseases and cancer. Aptamers are small single-stranded polynucleotides with a defined 3-dimensional conformation formed by Watson-Crick base-pairing. Due to their well-defined structure, they can be made to bind specific targets including isolated small molecules such as bacterial toxins or surface markers on cells with high affinity. Mercier, M C et al. (2017) Cancers (Basel) 9(6): E69; Ruscito, A et al. (2016) Front Chem 4:14. Aptamers are far smaller than antibodies, easier to produce and lack immunogenicity. Ray, P et al. (2013) Archivum Immunologiae et Therapiae Experimentalis 61(4): 255-271; Pei, X et al. (2014) Mol Clin Oncol 2(3): 341-348; Zhou, G et al. (2016) Oncotarget 7(12):13446-63. They are usually generated from a pool of up to 10¹⁵ random polynucleotides in an enrichment process that involves iterative binding, washing, and amplification steps. After each cycle, the aptamers with the highest target affinity are chosen for the next cycle. This leads to the selection of molecules with binding affinities in the nano- or even sub-nanomolar range after 10-12 cycles. This process is also known as systematic evolution of ligands by exponential enrichment (SELEX). Zhou, G et al. (2016). Analogous to antibodies, the first therapeutic aptamer approach aimed to block disease-related pathways through interaction with key proteins, receptors or metabolites. A prominent example is Macugen® (Pegaptanib; EyeTech Pharmaceuticals, Pfizer), the first FDA-approved aptamer therapeutic that entered the market in 2004. Macugen® is a 27 nucleotide-long RNA aptamer and is used in age-related macular degeneration (AMD), a serious eye disease causing blindness. AMD is characterized by abnormal formation of blood vessels due to elevated levels of growth factors. Macugen®'s target is VEGF₁₆₅ (isoform), a growth factor responsible for angiogenesis. Since this aptamer had only a short half-life due to fast renal clearance and degradation, it was bound to a 40 kDa PEG polymer to increase its overall size. In addition, some nucleotides were substituted with 2′-fluor-pyrimidine und 2′-O-methyl-purine to avoid degradation by nucleases. Biagi, C et al. (2014) Eur J Clin Pharmacol 70(12): 1505-12; Pozarowska, D et al. (2016) Cent Eur J Immunol 41(3): 311-316. In contrast to anti-VEGF antibodies (e.g., Bevacizumab, Avastin®; Roche), Macugen® has never been used or licensed for the treatment of cancer due to poor performance in systemic applications, probably due to compensation of effects by bypass pathways (e.g., PDGF-B). Alvarez, R H et al. (2006) Mayo Clin Proc 81(9): 1241-57. Following improvements made in more recent years, several attempts were made to use aptamers not only for targeting and blocking but also as carriers for cytotoxic agents. Bagalkot and co-workers developed an aptamer-doxorubicine complex by taking advantage of the agent's ability to intercalate into DNA. However, this complex suffered from poor loading efficiency and rapid systemic clearance. Bagalkot, V et al. (2006) Angew Chem Int Ed 45(48): 8149-8152. In 2010, a different approach was developed based upon docetaxel/cisplatin-loaded PLGA-PEG nanoparticles. These particles were guided to prostate cancer cells by functionalization with A10, an aptamer targeting a tumor cell membrane protein. This rather complex drug delivery system showed promising results at least in in vitro experiments. Kolishetti, N et al. (2010) Proc Natl Acad Sci USA 107(42): 17939-17944. Aptamers were further tested for the delivery of several nucleotide-based therapeutics such as siRNA (short interfering RNAs typically designed to suppress specific gene expression). Chu, T C et al. (2006) Nucleic Acids Res 34(10): e73. Despite the development of many different approaches utilizing the targeting ability of aptamers for tumor treatment, until present aptamers suffer from poor loading capacity, serum instability and fast renal clearance, all of which properties limit their clinical application. None of these aptamer-drug conjugates or complexes have entered clinical phase III or the market. Zhou et al. (2016).

To overcome the above-mentioned shortcomings and increase the drug to antibody/aptamer ratio (DAR) while simultaneously preserving antibody/aptamer affinity for the respective target, we developed a new strategy that utilizes a biocompatible, hydrophilic, non-degradable polymer as a carrier of active agents. The polymer is initially “loaded” with multiple molecules of active agent. Active agent is introduced into the polymer either during synthesis using active agent-conjugated monomers (therapeutic monomers) or subsequent to synthesis through functionalization. Typically, the active agent-containing polymer is subsequently coupled to a tumor-targeting moiety, e.g., a monoclonal antibody or an aptamer. Due to its high hydrophilicity, the polymer is capable of carrying even highly hydrophobic cytotoxic drugs while maintaining the pharmacokinetic properties of the respective antibody/aptamer. Polymer molecules can be made to carry a multiplicity (within limits, any desirable number) of active agent molecules.

The approach presented in this disclosure has the advantage that only one coupling site is needed to bind a multitude of active agent molecules to an antibody or aptamer molecule. By the use of site-specific coupling methods, e.g., enzymatic coupling reactions to peptide tags at the C-terminus of the heavy chains of an antibody, active agent-containing polymer will be located far away from the antibody's binding interface. With this approach, maximal affinity for the target tissue is preserved as well as a relatively homogenous product is received. The chosen linking strategy forms a stable peptide bond between copolymer and antibody/aptamer, which ensures high stability of the ADCs in the blood stream. Furthermore, the coupling of fully functionalized and characterized active agent-containing copolymer to antibody/aptamer in the last step is aimed at minimizing conformational stress on the sensitive binding proteins. In addition, the chosen design of the copolymers facilitates the coupling of two or more different active agents to the same molecule, enabling combination therapies. Once active agent (also referred to as cytotoxic drug or the toxic payload in the cancer context) is released inside a targeted cell, e.g., a tumor cell, and the targeting moiety (e.g., an antibody or an aptamer) is degraded, the relatively small copolymer is believed to be removed from the body by renal clearance.

### SUMMARY OF THE INVENTION

The present disclosure relates to a copolymer molecule containing multiple molecules of an active agent as well as to methods for making this copolymer. The copolymer is made by polymerization of a reaction mixture comprising (1) one or more (types of) polymerizable principal monomers, which monomers are characterized as having at least one vinylic group and not containing an amino acid residue, (2) one or more (types of) co-principal monomers of formulae I and/or II in which at least one of Y and Z is H, (3) an agent for controlling radical polymerization, which agent is preferably a RAFT agent, and (4) an initiator system for generating free radical species. The reaction mixture can optionally further comprise one or more co-principle monomers of any of formulae III to X. The latter polymerization yields a copolymer that can be functionalized with multiple active agent molecules. This functionalization occurs at free alpha-amino or alpha-carboxy groups of co-principal monomer units.

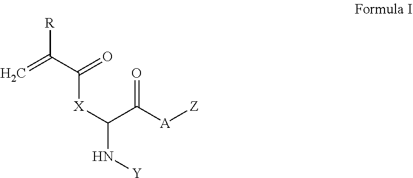

wherein R is —H, —CH₃, —CH₂—CH₃ or —(CH₂)₂—CH₃; X is —NH(CH₂)₄—, —NH(CH₂)₃—, —O—C₆H₄—CH₂—, —O—CH₂—, —O—CH(CH₃)—, —S—CH₂— or —NH—C₆H₄—CH₂—; Y is H or —CO—CₙH₂ₙ₊₁ (with n=1-8); Z is H (if A is —O—) or —CₙH₂ₙ₊₁ (with n=1-8); and A is —O— or —NH—.

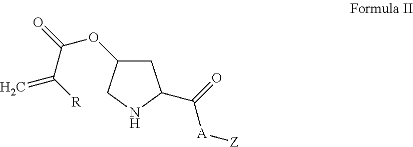

wherein: R is —H, —CH₃, —CH₂—CH₃ or —(CH₂)₂—CH₃; Z is H (if A is O) or —CₙH₂₊₁ (with n=1-8); and A is —O— or —NH—.

Depending on the structure of the active agent, active agent molecules may be bound directly or indirectly via linker structures to alpha-amino or alpha-carboxylic groups of co-principle monomers in the copolymer. The latter linker should be stable during storage and in the blood stream to avoid unintended release of cytotoxic drug. The linker may be capable of being cleaved by specific intracellular enzymes or may be of a “non-degradable” type and only destroyed in the harsh environments of lysosomes and peroxisomes.

The copolymer molecule containing multiple molecules of an active agent can be further functionalized with a cell type-specific or a tissue type-specific targeting moiety. Potential targeting moieties are, but are not limited to, monoclonal antibodies, antibody fragments, nano-bodies (single-domain-antibodies), DARPins (designed ankyrin repeat proteins), peptide hormones, proteins binding to proteins expressed on the tumor cell surface. DNA- or RNA-based aptamers, or small molecules capable of binding to cell surface receptors that are known to be over-expressed in tumor cells, e.g., folic acid or biotin. The covalent attachment of the targeting moiety is carried out in a site-specific manner, typically involving a reactive group in the copolymer's head group (that typically is introduced via a RAFT agent). Suitable coupling strategies include enzyme-catalyzed reactions with peptide tags, e.g., sortase-mediated coupling, aldehyde tags, or transglutaminase tags, or the so-called “click” reaction between copolymer and targeting moiety. The latter process may be achieved by integration of reactive, non-canonical (unnatural) amino acids into the targeting moiety during synthesis or post synthesis. Sortase-mediated coupling and transglutaminase-mediated coupling are preferred methods. In the former mechanism, the targeting moiety is modified to contain a sortase motif. The copolymer molecule carrying multiple molecules of an active agent can be made a target for sortase-mediated transpeptidation by introduction of an oligo-glycine stretch at the copolymer's head group. This may be conveniently achieved during polymerization in which a conventional RAFT agent is replaced with a derivatized RAFT agent containing 2-8 glycine residues. In case of a transglutaminase-mediated reaction, the co-polymer's head group introduced by a suitable chain transfer agent may comprise a peptide motif containing a reactive lysine (or glutamine) residue or a non-peptide motif e.g., a linker structure containing a terminal amino group. The latter head group modification may especially be used in combination with microbial transglutaminases, which are known to accept non-peptide motifs with high turnover rates.

In different embodiments the here presented enzymatic reactions may also be used to modify the cell- or tissue-type specific targeting moiety site specifically with a reactive group, e.g., a so-called “click-reactive” group (such as azide for [3+2] cycloaddition or tetrazine for [4+2] cycloaddition) which is subsequently used to bind a copolymer of this disclosure containing the “counterpart” (e.g. an alkyne in case of a [3+2] cycloaddition or a strained alkene for a [4+2] cycloaddition) of the click reaction in its head group. The above-mentioned reactive parts of the click reaction are meant to be interchangeable.

In another embodiment, in which the active agent is unstable such as is the case for molecules containing a short-lived radioisotope, the copolymer made as described above is first functionalized with a cell type- or tissue type-specific targeting moiety using one of the above-described methods, e.g., sortase-mediated or transglutaminase-mediated coupling. Prior to therapeutic use, the targeting moiety-copolymer conjugate is then loaded with active agent, whereby active agent molecules are bound either directly or indirectly via a linker structure to free alpha-amino or carboxylic groups of the copolymer.

A copolymer containing multiple molecules of an active agent can also be made in two successive polymerization reactions. For example, the first polymerization reaction is carried out in a first reaction mixture comprising one or more (types of) polymerizable principal monomers not containing an amino acid group, a RAFT agent, and an initiator system for generating free radical species, the polymerization yielding a RAFT pre-polymer. The second polymerization reaction is carried out in a second reaction mixture comprising the RAFT pre-polymer of the first polymerization reaction, one or more (types of) co-principle monomers of formulae I and/or II, and an initiator system for generating free radical species. The reaction can optionally include one or more (types of) co-principal monomers of any of formulae III-X, and/or one or more polymerizable principal monomers not containing an amino acid group.

In a more particular embodiment, a copolymer that contains multiple active agent molecules is made by polymerization of a reaction mixture comprising (1) one or more (types of) polymerizable principal monomers, which monomers are characterized as having at least one vinylic group and not containing an amino acid residue, (2) one or more (types of) co-principal monomers of formula I and/or formula II in which at least one of Y and Z is H, (3) optionally one or more (types of) co-principal monomers of formulae III to X (4) a RAFT agent containing a monodisperse spacer (i.e., a spacer of uniform size) of 5-25 units, and (5) an initiator system for generating free radical species.

In different embodiments, a copolymer that contains multiple active agent molecules is made by polymerization of a reaction mixture comprising (1) one or more (types of) polymerizable principal monomers, which monomers are characterized as having at least one vinylic group and not containing an amino acid residue, (2) one or more (types of) co-principal monomers of formulae III to X, (3) optionally one or more (types of) co-principal monomers of formula I and/or formula II, (4) an agent for induction of controlled radical polymerization, which agent is preferably a RAFT agent, and (5) an initiator system for generating free radical species.

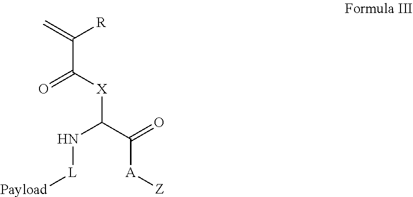

wherein R is —H, —CH₃, —CH₂—CH₃ or —(CH₂)₂—CH₃; X is —NH(CH₂)₄—, —NH(CH₂)₃—, —O—C₆H₄—CH₂—, —O—CH₂—, —O—CH(CH₃)—, —S—CH₂— or —NH—C₆H₄—CH₂—; Z is H (if A is —O—) or —CₙH₂ₙ₊₁ (with n=1-8); payload refers to an active agent; L is a linker and A is —O— or —NH—.

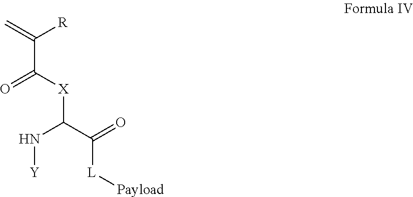

wherein R is —H, —CH₃, —CH₂—CH₃ or —(CH₂)₂—CH₃; X is —NH(CH₂)₄—, —NH(CH₂)₃—, —O—C₆H₄—CH₂—, —O—CH₂—, —O—CH(CH₃)—, —S—CH₂— or —NH—C₆H₄—CH₂—; Y is H or —CO—CₙH₂ₙ₊₁ (with n=1-8); payload refers to an active agent; and L is a linker.

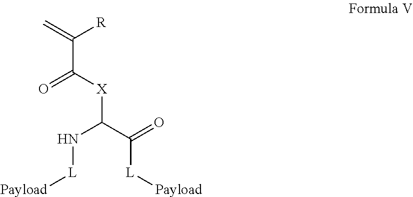

wherein R is —H, —CH₃, —CH₂—CH₃ or —(CH₂)₂—CH₃; X is —NH(CH₂)₄—, —NH(CH₂)₃—, —O—C₆H₄—CH₂—, —O—CH₂—, —O—CH(CH₃)—, —S—CH₂— or —NH—C₆H₄—CH₂—; payload refers to an active agent and L is a linker, whereby the linkers used for functionalizing the alpha-amino and carboxy groups do not need to be identical.

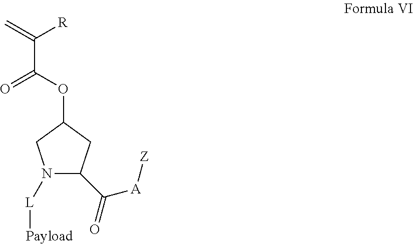

wherein R is —H, —CH₃, —CH₂—CH₃ or —(CH₂)₂—CH₃; X is —NH(CH₂)₄—, —NH(CH₂)₃—, —O—C₆H₄—CH₂—, —O—CH₂—, —O—CH(CH₃)—, —S—CH₂— or —NH—C₆H₄—CH₂—; Z is H (if A is —O—) or —CₙH₂ₙ₊₁ (with n=1-8); payload refers to an active agent; L is a linker and A is —O— or —NH—.

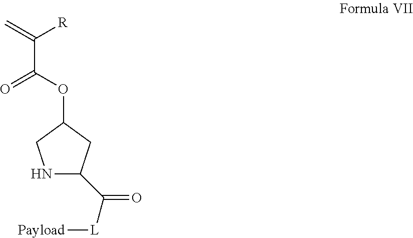

wherein R is —H, —CH₃, —CH₂—CH₃ or —(CH₂)₂—CH₃; X is —NH(CH₂)₄—, —NH(CH₂)₃—, —O—C₆H₄—CH₂—, —O—CH₂—, —O—CH(CH₃)—, —S—CH₂— or —NH—C₆H₄—CH₂—; payload refers to an active agent; and L is a linker.

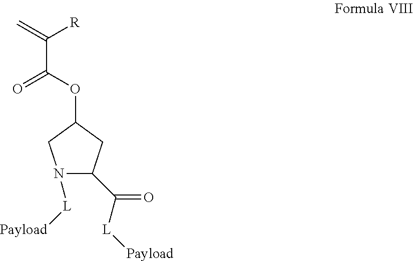

wherein R is —H, —CH₃, —CH₂—CH₃ or —(CH₂)₂—CH₃; X is —NH(CH₂)₄—, —NH(CH₂)₃—, —O—C₆H₄—CH₂—, —O—CH₂—, —O—CH(CH₃)—, —S—CH₂— or —NH—C₆H₄—CH₂—; payload refers to an active agent and L is a linker, whereby the linkers used for functionalizing the alpha-amino and carboxy groups do not need to be identical.

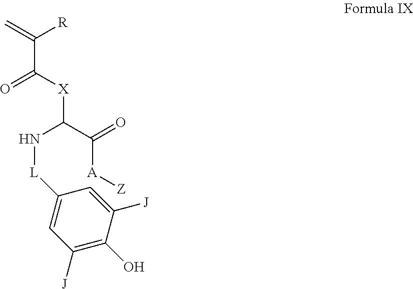

wherein R is —H, —CH₃, —CH₂—CH₃ or —(CH₂)₂—CH₃; X is —NH(CH₂)₄—, —NH(CH₂)₃—, —O—C₆H₄—CH₂—, —O—CH₂—, —O—CH(CH₃)—, —S—CH₂— or —NH—C₆H₄—CH₂—; Z is H (if A is —O—) or —CₙH₂ₙ₊₁ (with n=1-8); L is a linker; J is H or an radioactive iodine nucleus and A is —O— or —NH—.

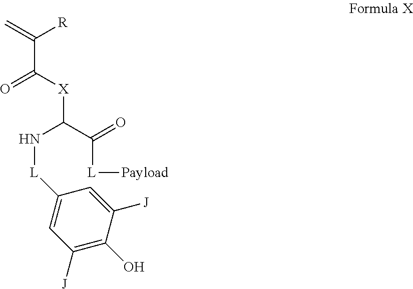

Wherein R is —H, —CH₃, —CH₂—CH₃ or —(CH₂)₂—CH₃; X is —NH(CH₂)₄—, —NH(CH₂)₃—, —O—C₆H₄—CH₂—, —O—CH₂—, —O—CH(CH₃)—, —S—CH₂— or —NH—C₆H₄—CH₂—; J is H or an radioactive iodine nucleus. Payload refers to an active agent; and L is a linker, whereby the linkers used for functionalizing the alpha-amino and carboxy groups do not need to be identical.

In a more particular embodiment, a copolymer that contains multiple active agent molecules is made by polymerization of a reaction mixture comprising (1) one or more (types of) polymerizable principal monomers, which monomers are characterized as having at least one vinylic group and not containing an amino acid residue, (2) one or more (types of) co-principal monomers of formulae III to X, (3) optionally one or more (types of) co-principal monomers of formula I and/or formula II in which at least one of Y and Z is H, (4) a RAFT agent containing a monodisperse spacer of 5-25 units, and (5) an initiator system for generating free radical species.

A copolymer containing multiple molecules of an active agent can also be made in two successive polymerization reactions. For example, the first polymerization reaction is carried out in a first reaction mixture comprising one or more (types of) polymerizable principal monomers not containing an amino acid group, a RAFT agent, and an initiator system for generating free radical species, the polymerization yielding a RAFT pre-polymer. The second polymerization reaction is carried out in a second reaction mixture comprising the RAFT pre-polymer of the first polymerization reaction, one or more (types of) co-principle monomers of formulae III to X, and an initiator system for generating free radical species. The reaction can optionally include one or more (types of) co-principal monomers of formula I and/or formula II and/or one or more polymerizable principal monomers not containing an amino acid group.

The latter copolymer molecules containing multiple molecules of an active agent can be further functionalized with a cell type or tissue type-specific targeting moiety as has been described for the initial embodiment.

The parenthetical term “types of” has been included to make it clear that expressions such as “one or more polymerizable co-principal monomers” do not refer to one or more molecules of the monomer but to amounts of one or more chemically different monomers of the formula (e) in question.

In any of the above-described copolymers containing multiple molecules of an active agent, the total amount of monomers of any of formula I to formula X preferably ranges from 1% (mol) to 49.9% (mol) of all monomers contained in the copolymer. More preferably, the total amount of monomers of formula I to formula X ranges from 1% (mol) to 35% (mol) of all monomers contained in the copolymer. Even more preferably, the total amount of monomers of formula I to formula X ranges from 1% (mol) to 20% (mol) of all monomers contained in the copolymer. Most preferably, the total amount of monomers of formula I to formula X ranges from 5% (mol) to 15% (mol) of all monomers contained in the copolymer.

In any of the above-described copolymers containing multiple molecules of an active agent, the copolymers have average molecular weights of 5,000 Daltons to 100,000 Daltons. More preferably, the copolymers have average molecular weights of 6,000 Daltons to 60,000 Daltons. Most preferably, the copolymers have average molecular weights of 6,000 Daltons to 20,000 Daltons.

In any of the above-described copolymers containing multiple molecules of an active agent, at least 80% (w) of the copolymer molecules have an average molecular weight of 5,000 Daltons to 100,000 Daltons. More preferably, at least 80% (w) of the copolymer molecules have an average molecular weight of 6,000 Daltons to 60,000 Daltons. Most preferably, at least 80% (w) of the copolymer molecules have an average molecular weight of 6,000 Daltons to 20,000 Daltons.

As discussed above, polymerization mixtures for the preparation of any of the above-described copolymers containing multiple molecules of an active agent can comprise a RAFT agent that carries a reactive group which can be used for functionalization of the copolymer with a cell type- or tissue type-specific targeting moiety. The latter reactive group can be a thiol, an aldehyde, an alkyne, an azide, an amine, a carboxyl, an ester, a diazirine, a phenyl azide, a thioester, a diazo, a Staudinger reactive phosphinoester (or phosphinothioester), a hydrazine, an oxime, an acrylate to perform aza-Micheal ligations, or a motif capable of being used in an enzymatic coupling reaction. The motif can be an oligo-glycine comprising 2-8 amino acids, which peptide motif enables sortase-mediated coupling reactions, a transglutaminase reactive substrate, an aldehyde tag or an autocatalytic intein sequence.

In other specific embodiments, RAFT agent is inactivated once polymerization and/or functionalization has been completed, whereby the elimination of the RAFT group is performed by thermal treatment, reaction with suitable amines (aminolysis), or a new reaction with an initiator molecule in the presence of a phosphorus oxoacid or with excess of initiator without phosphorus oxoacid.

In any of the above-described copolymers containing multiple molecules of an active agent, the active agent can be a microtubule inhibitor, an intercalating agent, an alkylating agent, an antimetabolite, a hormone or hormone receptor modulation agent, a tyrosine kinase inhibitor, a polynucleotide-based drug capable of interfering with a gene or its respective messenger RNA, a protein-based bacterial toxin, an enzyme suitable for prodrug therapy (ADEPT concept), or a radioisotope. The active agent can also be a tracer molecule including a small molecule fluorophore, a protein/peptide-based fluorophore, a near infrared (NIR) fluorescent probe, a bioluminescent probe, a radiocontrast agent, or a radioisotope.

The present disclosure also relates to pharmaceutical compositions comprising an effective amount of a copolymer containing multiple molecules of an active agent as detailed above and a carrier. Depending on the nature of the active agent, these compositions may be used in the treatment of various cancers or of other diseases/conditions.

The present disclosure also encompasses methods of treatment of different types of cancers or other diseases and conditions comprising administration of a pharmaceutical composition comprising an effective amount of a copolymer containing multiple molecules of an active agent of the present disclosure (also referred to herein as “active moiety”). Within the scope of the present disclosure are also uses of pharmaceutical compositions comprising an effective amount of a copolymer containing multiple molecules of an active agent of the present disclosure for the treatment of a cancer or another disease or condition in a subject, comprising administering to the subject an effective amount of a copolymer containing multiple molecules of an active agent.

### DETAILED DESCRIPTION OF THE INVENTION

Unless otherwise defined, all terms shall have their ordinary meaning in the relevant art. The following terms are defined and shall have the following meanings:

As used herein, “pharmaceutically acceptable carrier or excipient” is intended to include any and all solvents, dispersion media, coatings, antibacterial and antifungal agents, isotonic and absorption delaying agents, and the like, compatible with pharmaceutical administration, such as sterile pyrogen-free water. Suitable carriers are described in Remington's Pharmaceutical Sciences (Mack Publishing Co., Easton, Pa., 19th ed. 1995), a standard reference text in the field, which is incorporated herein by reference. Non-limiting examples of materials which can serve as pharmaceutically acceptable carriers are sugars such as lactose, glucose and sucrose; cyclodextrins such as alpha-, beta- and gamma-cyclodextrins; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Also encompassed are emulsifiers/surfactants such as cremophor EL and solutol HS15, lecithin and phospholipids such as phosphatylcholine. Liposomes may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “subject” as used herein refers to a mammalian subject. Preferably, the subject is a human subject.

The term “active moiety” relates to a copolymer containing multiple molecules of an active agent of the present disclosure (which copolymer may be further functionalized with a cell type-specific or tissue type-specific targeting moiety).

The term “cell type-specific or tissue type-specific targeting moiety” in the context of this disclosure refers to a molecule that binds to a surface marker on cells of a specific type or on cells of a particular tissue with an avidity, that renders it useful for the delivery to the cells of a cargo active agent. It can be a monoclonal antibody, a single-domain, variable fragment of an antibody chain, a single-chain antibody, a DARPin (Designed Ankyrin Repeat Protein), a DNA- or RNA-based aptamer, a peptide-based aptamer, a peptide or protein capable of binding a cell surface marker, a hormone, or a small molecule capable of binding a cell surface marker.

A “tracing molecule” is defined as a molecule that is capable of producing a readout signal in a diagnostic or scientific application. It can be a small molecule fluorophore, a protein/peptide-based fluorophore, a near infrared (NIR) fluorescent probe, a bioluminescent probe, a radiocontrast agent, or a radioisotope.

By an “effective amount” of an active moiety of the disclosure is meant an amount of the active moiety which, when administered once or multiple times over the course of a treatment, confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of an active moiety of the disclosure is an amount of the active moiety that comprises an active agent preferably in an amount ranging from about 0.01 mg/kg body weight of a subject to about 50 mg/kg body weight, and more preferably from about 0.1 to about 30 mg/kg body weight. Effective doses will also vary depending on route of administration, as well as the possibility of co-usage with other agents. It will be understood, however, that the total daily usage of the active moiety and pharmaceutical compositions of the present disclosure will be decided by the attending physician within the scope of sound medical judgment. The specific effective dose level for any particular patient will depend upon a variety of factors including the disorder being treated and the severity of the disorder; the activity of the specific active agent employed; the specific composition employed; the age, body weight, general health, sex and diet of the patient; the time of administration, route of administration and rate of excretion of the specific active moiety employed; the duration of the treatment; drugs used in combination or contemporaneously with the specific active moiety employed; and like factors well known in the medical arts. It is noted that when used in the context of prophylaxis or prevention, an “effective amount” of an active moiety of the disclosure is meant to be an amount of the active moiety which, when administered once or multiple times over the course of a treatment, confers a desired prophylactic effect on the treated subject.

The term “active agent” means a therapeutically active substance, which is bound to copolymers of this disclosure. In the context of cancer therapy, the active agent typically is a cytotoxic substance/molecule. Example cytotoxic substance/molecules include microtubule inhibitors such as monomethyl auristatin E (MMAE) or emtansine (DM1), intercalating drugs, e.g., doxorubicin, alkylating agents such as cyclophosphamide (CP), antimetabolites such as 5-fluoruracil (5-FU), hormones or hormone receptor modulation agents such as tamoxifen citrate, tyrosine kinase inhibitors such as Afatinib or Bosutinib, peptide-based toxins, e.g. α-amanitin, immune checkpoint inhibitors such as Nivolumab® or Pembrolizumab®, enzymes suitable for antibody-directed enzyme prodrug therapy (ADEPT), polynucleotide-based drugs capable of interfering with a gene(s) or its respective messenger RNA (siRNA, microRNA or antisense-RNA) and radioisotopes such as, but not limited to, fluor-18, copper-64, gallium-68, zirconium-89, indium-111, iodine-123 (diagnostic application) or strontium-89, yttrium-90, iodine-131, samarium-153, lutetium-177, radium-223 and actinium-225 (therapeutic application).

Radioisotopes are coupled either to a co-principle monomer before polymerization or to a copolymer after polymerization. Chelating agents that are covalently coupled to the co-principle monomers before polymerization or to a copolymer after polymerization can be used to immobilize radioisotopes. Chelating agents include, but are not limited to, (1,4,7,10)-Tetraazacyclododecane-1,4,7,10-tetraacetic acid [DOTA], 2,2′,2″-(10-(2,6-Dioxotetrahydro-2H-pyran-3-yl)-1,4,7,10-tetraazacyclododecane-1,4,7-triyl)triacetic acid [DOTA-GA], 1,4,7-Triazacyclononane-N,N′,N″-triacetic acid [NOTA], 1,4,8,11-Tetraazacyclotetradecane-1,4,8,11-tetraacetic acid) [TETA] and Diethylene-triamine-pentaacetic anhydride [DTPA].

The term “active agent” in the context of this disclosure further encompasses substances capable of overcoming tumor cell resistance, e.g., by inhibiting an anti-apoptotic factor such as Bcl-2 or targeting a cellular efflux pump (such as the MDR-1 transporter), or anti-inflammatory substances including corticosteroids, glucocorticoids and nonsteroidal anti-inflammatory drugs (e.g., prostaglandins) that are useful for reducing inflammation-related therapy side effects.

A “monomer” means a low molecular weight compound that can be polymerized. For co-principal monomers of formula I or II, or for principal monomers, low molecular weight typically means a molecular weight of less than 800 Daltons. For co-principal monomers of formulae III-X, low molecular weight typically means a molecular weight of less than 1500 Daltons. When referred to in the context of a copolymer, the term “monomer” refers to the smallest building blocks of the copolymer.

The terms “RAFT agent” and “RAFT process” involve conventional free radical polymerization of a monomer in the presence of a suitable chain transfer agent (CTA). Commonly used RAFT agents include thiocarbonylthio compounds such as dithioesters, dithiocarbamates, trithiocarbonates and xanthates, which agents mediate the polymerization via a reversible chain-transfer process. Chiefari, J. et al. (1998) Macromolecules 31(16): 5559-62.

The term “pre-polymer” relates to a short polymer headed by a RAFT agent and comprising 10-25 units of a hydrophilic principal monomer, e.g., dimethyl-acrylamide. Such pre-polymers represent water-soluble macro-RAFT agents that are used in a second polymerization reaction to synthesize copolymers of principal and co-principal monomers in an aqueous environment.

The terms “substrate, motif or tag” or “reactive substrate, motif or tag” are used interchangeably to relate to chemical structures being able to take part in an enzymatically catalyzed reaction. These chemical structures are recognized by the active center of an enzyme and may intermediately form a covalent or electrostatic enzyme-substrate complex before the enzymatic catalyzed reaction takes place. In the context of the present disclosure, these reactions are often used to mediate the covalent attachment of a copolymer of this disclosure to a tumor cell or tissue-specific targeting moiety. Typical substrates, motifs and tags are defined sequences of amino acids or peptides, reactive functional groups like amino, thiol or carboxyl groups or unsaturated carbon bonds in a flexible spacer region of the copolymer's head group.

The term “antibody-drug conjugate”, abbreviated “ADC”, represents a combination of an antibody that targets cell type- or tissue type-specific antigens (including tumor antigens) with a drug molecule or a multitude of drug molecules wherein the drug molecules are covalently attached to the antibody. In the context of the present disclosure, ADC refers to a conjugate of a cell type- or tissue type-specific antigen-targeting antibody with a copolymer containing multiple molecules of an active agent of the present disclosure. As discussed, the copolymer of the present disclosure carries a multitude of active agent molecules or a combination of different active agent molecules that are bound, via a linker or directly, to alpha-amino and alpha-carboxy groups in co-principal monomers.

The term “aptamer” is defined as follows: aptamers are oligonucleotide or peptide molecules that bind to a specific target molecule. Aptamers are usually created by selecting them from a large random sequence pool in an iterative enrichment process to identify the aptamer sequence with the highest target affinity. This process is also known as “systematic evolution of ligands by exponential enrichment (SELEX)”. More specifically, aptamers can be classified as DNA, RNA, xeno nucleic acid (XNA) (a synthetic alternative to natural nucleic acids that differs in the sugar backbone) or peptide aptamers. Aptamers consist of (usually short) strands of oligonucleotides or sequences of amino acids. The oligonucleotide sequence can thereby be formed of one kind of nucleotide, e.g., DNA, or a combination of different nucleotide types, e.g., DNA, RNA and or specially designed so called “locked-nucleotides” having their ribose moiety modified with an extra bridge connecting the 2′ oxygen and 4′ carbon. Aptamers in this disclosure also means peptide aptamers consisting of one (or more) short peptide domains.

The term “aptamer-drug conjugate” means a combination of an aptamer with an active agent molecule or different active agent molecules. In the context of the present disclosure, the active agent molecules are attached to copolymers either prior to or subsequent to the coupling of the copolymer to the aptamer.

The term “enhanced permeability and retention (EPR) effect” is used to describe the abnormal molecular and fluid transport dynamics in tumor tissue, especially for macromolecular drugs. Molecules of certain sizes (typically liposomes, nanoparticles, and macromolecular drugs) tend to accumulate in tumor tissue at higher levels than in normal tissues. The general explanation that is given for this phenomenon is that, in order for tumor cells to grow quickly, they must stimulate the production of blood vessels. The newly formed tumor vessels are usually abnormal in form and architecture and are permeable for molecules of higher molecular weight. Furthermore, tumor tissues usually lack effective lymphatic drainage so that, once a molecule has entered the tumor tissue, it is not effectively removed from this tissue.

The term “side chain-linked amino acid” in the context of a co-principal monomer means that an amino acid is covalently linked through its side chain (e.g., through an ester or amide linkage) to a moiety containing an acryloyl group. Monomers of formulae I to X contain side chain-linked amino acids.

The terms “principal monomer” and “co-principal monomer” are used mainly to facilitate the description of the invention. Principal monomers refer to monomers that do not include an amino acid, and co-principal monomers refer to monomers that do contain an amino acid.

Copolymers containing the latter principal and co-principle monomers are also generically referred to as “Cellophil copolymers”, the term “Cellophil” serving to indicate the presence in the copolymers of monomers containing side chain-linked amino acids (that may be further functionalized as, e.g., in formulae III-X). Side chain-linked amino acids include lysine (K), tyrosine (Y), serine (S), threonine (T), cysteine (C), 4-hydroxyproline (HO—P), ornithine (ORN) and 4-amino-phenylalanine (HOX). The amino acids can be the L or the D forms, or racemic mixtures. In the copolymers, a single type of side chain-linked amino acid or multiple types of side chain-linked amino acids may be present. For example, a copolymer can comprise both acryloyl-L-lysine (AK) and acryloyl-L-threonine (AT). For the sake of clarity, all monomers described by formulae I-X include a side chain-linked amino acid (functionalized or not functionalized). The amino acid-containing copolymers of this disclosure comprise one or more polymerizable principal monomers, which monomers are characterized as having at least one vinylic group but not containing an amino acid residue, one or more co-principal monomers according to any of formula I to formula X (including co-principal monomers reading on two or more of the latter formulae).

Preferably, the co-principal monomers are present in a polymerization mixture in an amount between 1% (mol) and 49.9% (mol) of all monomers contained in the copolymer. More preferably, the co-principal monomers are present in a polymerization mixture in an amount between 1% (mol) and 35% (mol), even more preferably between 1% (mol) and 20% (mol), and most preferably between 5% (mol) and 15% (mol) of all monomers contained in the copolymer.

The synthesis of monomers containing side chain-linked amino acids was described previously. Zbaida, D et al. (1987) Reactive Polymers, Ion Exchangers, Sorbents 6(2-3): 241-253. Such monomers can be prepared by reacting the amino acid copper complex of lysine, tyrosine, serine, threonine, cysteine, ornithine, 4-amino-phenylalanine or 4-hydroxyproline with either acryloyl chloride, methacryloyl chloride, ethyl-acryloyl chloride or propyl-acryloyl chloride, followed by treatment with a stream of hydrogen sulfide gas or an acidic solution of sodium sulfide to yield the unprotected monomer. Protocols are disclosed under examples.

In particular embodiments, the principle monomers are derivatives of acrylamide and include dimethyl-acrylamide, N-isobutyl-acrylamide, N-tert. butyl-acrylamide, N-hydroxyethyl-acrylamide, N-(2-Hydroxypropyl)-acrylamide, N-(3-Hydroxypropyl)-acrylamide, N-(3-Hydroxypropyl)-methacrylamide, N-(2-Hydroxypropyl)-methacrylamide, N-(3-Aminopropyl)-acrylamide hydrochloride, or N-(3-Aminopropyl)-methacrylamide hydrochloride.

In other particular embodiments, the principle monomers are derivatives of acrylic acid including meth-acrylic acid 2-hydroxyethyl-acrylate, 2-hydroxypropyl-acrylate, 3-hydroxypropyl-acrylate, 2-hydroxy-1-methylethyl-acrylate, 2-aminoethyl acrylate hydrochloride, 3-hydroxypropyl-methacrylate, 2-hydroxy-1-methylethyl-methacrylate, 2-hydroxyethyl-methacrylate, 2-hydroxypropyl-methacrylate and 2-aminoethyl methacrylate hydrochloride.

Copolymers comprising one or more types of co-principal monomers of formulae I to X and one or more types of principal monomers are typically prepared in a radical polymerization reaction. It is important that copolymers of this disclosure have a narrow size distribution because in various therapies, in particular in cancer therapies, the drug load has to be precisely controlled. If it is not carefully controlled, over-dosing or under-dosing effects may be encountered. To obtain copolymers with a narrow size distribution, the number of free radicals in the polymerization process has to be controlled. This can be achieved by the use of polymerization techniques including atom transfer radial polymerization (ATRP), nitroxide-mediated polymerization (NMP) or reversible addition-fragmentation-chain transfer polymerization (RAFT polymerization). RAFT is the most preferred technique for the copolymers described herein as it is compatible with a broad spectrum of monomers, especially acrylics, and can be easily performed in aqueous systems. Furthermore, RAFT polymerization can be used for the synthesis of block copolymers. In addition, the RAFT group can be used to add a reactive moiety to a polymer's head group (e.g., for conjugation with an antibody or aptamer). The RAFT technology was invented by a research group of the Commonwealth Scientific and Industrial Research Organization (CSIRO). Chiefari et al. (1998). Control of the chain size distribution is achieved via chain transfer reactions from the growing polymer chain to a chain transfer agent. A so-called RAFT agent forms an intermediate and is able to fragment into a radical on the propagating chain (designated as R-group) and a stabilizing moiety (designated as Z-group). As a consequence, the number of radicals is limited, and all growing polymer chains have a similar likelihood of propagation, resulting in copolymers with a narrow size distribution. Typical poly-dispersion-indices (PDIs) [defined as Mw/Mn, where Mw is the weight-average molar mass and Mn is the number-average molar mass of the polymer] obtained in RAFT polymerizations are in the range of 1.05 to 1.4. Suitable RAFT agents are thiocarbonylthio compounds. Thiocarbonylthio compounds can be divided into four main classes, i.e., dithiobenzoates, trithiocarbonates, dithiocarbamates, and xanthates.

A typical polymerization mixture of this disclosure comprises therefore principal and co-principal monomers, a RAFT agent, and a radical initiator. The mixture is then poured into a suitable container or mold, wherein polymerization is induced. Initiators can be thermal initiators, e.g., VA-044 that is destabilized at elevated temperature to produce reactive radicals, redox initiators or photo initiators. Preferred redox initiators for polymerization in aqueous solution are peroxides, e.g., ammonium persulfate or potassium persulfate in combination with sodium thiosulfate, or azo-type compounds, for instance 2,2′-Azobis[2-(2-imidazolin-2-yl)propane]dihydrochloride or 4,4′-Azobis(4-cyanovaleric acid). For polymerization reactions in non-aqueous solvents, initiators/catalysts of the azo-type, e.g., Azobis(isobutyronitrile (AIBN), 1,1′-Azobis(cyclohexane-1-carbonitrile), 2,2′-Azobis(4-methoxy-2,4-dimethylvaleronitrile) are preferred. Polymer-modified azo-type initiators e.g., (polydimethylsiloxane, polyethylenglycol) can also be utilized. The above-mentioned initiators are usually destabilized at higher temperatures leading to the formation of reactive radicals.

Alternatively, the monomers can be photo-polymerized in a container or mold that is transparent to radiation of a wavelength capable of initiating polymerization of the vinylic or acrylic monomers. Suitable photoinitiator compounds could be from type I, e.g., α-amino alkylphenones, or type II, e.g., benzophenones. Photosensitizers that permit the use of longer wavelengths can also be utilized. Depending on the initiator compound used, polymerization is initiated by heating, radiation or addition of a catalyst.

In some embodiments of this disclosure, it is useful to synthesize a macro-RAFT or pre-polymer composed of 10-25 monomer units of a hydrophilic principal monomer prior to the polymerization of the copolymers (containing a mixture of principal and co-principal monomers). With this, the hydrophilicity of the often-hydrophobic RAFT agents can be enhanced, facilitating polymerization reactions in an aqueous environment.

In other embodiments, the RAFT agent itself is chemically modified by the integration of a water-soluble monodisperse polyethylene glycol (PEG) spacer of 5-25 units. The modified RAFT agent exhibits an improved water solubility and enables the synthesis of hydrophilic amino acid-containing copolymers in one polymerization step.

Since the RAFT agent is known to be unstable in the presence of amines and is responsible for a strong odor of the obtained copolymers, it should usually be inactivated once the polymerization and functionalization process is completed. Preferred methods for RAFT group inactivation in this disclosure are reactions with nucleophiles, thermal elimination, or a second reaction with an initiator in combination with a proton-donating agent or an excess of a functionalized initiator.

Since the copolymers of this disclosure are intended to be used for drug delivery in a patient, it is generally preferable to purify the copolymers after polymerization. This step removes potentially harmful ingredients including residual initiators, monomers or catalysts. Preferred purification methods for copolymers of this invention are dialysis, tangential flow filtration and capillary ultrafiltration.

It is noted that defining useful parameter values does not require undue effort both because the number of parameters is limited and preferred ranges of some parametric values are known. The level of co-principal monomers in an amino acid-containing copolymer will preferably be between 1% (mol) and 49.9% (mol), more preferably between 1% (mol) and 35% (mol), even more preferably between 1% (mol) and 20% (mol) and most preferably between 5% (mol) and 15% (mol) of all monomers present in the polymerization mixture. The average molecular weight of the amino acid-containing copolymer (without therapeutic payload) will generally be between 5,000 and 100,000 Daltons, preferably between 6,000 and 60,000 Da, and most preferably between 6,000 and 20,000 Da.

Once copolymerization and purification have been completed, a copolymer of this invention comprising co-principal monomers of formula I and/or II is ready for functionalization with active agent molecules and/or a cell type-specific or a tissue type-specific targeting moiety (e.g., an antibody). This functionalization results in the establishment of covalent bonds between copolymer and active agent molecules and/or targeting moiety. In the case of certain active agents, e.g., certain radioisotopes, a chelating agent is covalently bound to the copolymer, and the active agent is held by the chelating agent.

In particular embodiments, an active agent (here a cytotoxic drug or molecule used in cancer therapy) can be a microtubule inhibitor such as monomethyl auristatin E (MMAE) or emtansine (DM1); an intercalating drug, e.g., doxorubicin; an alkylating agent such as cyclophosphamide (CP); an antimetabolite such as 5-fluoruracil (5-FU); a hormone or hormone receptor modulation agent such as tamoxifen citrate; a tyrosine kinase inhibitor such as Afatinib or Bosutinib; a peptide-based toxin, e.g., α-amanitin; an immune checkpoint inhibitor such as Nivolumab® or Pembrolizumab®; an enzyme suitable for antibody-directed enzyme prodrug therapy (ADEPT); a polynucleotide-based drug capable of interfering with a gene(s) or its respective messenger RNA, siRNA, microRNA or antisense-RNA; or a radioisotope such as, but not limited to, fluor-18, copper-64, gallium-68, zirconium-89, indium-111, iodine-123 (diagnostic application) or strontium-89, yttrium-90, iodine-131, samarium-153, lutetium-177, radium-223 and actinium 225 (therapeutic application).

In yet other particular embodiments, the active agents are a combination of a cytotoxic drug and a drug being capable of overcoming tumor cell resistance, for instance by inhibiting an anti-apoptotic factor such as Bcl-2 or targeting a cellular efflux pump (such as the MDR-1 transporter).

The afore-mentioned active agents are nonlimiting examples of agents and agent classes that are compatible with the copolymers of this disclosure, and someone skilled in art may use variants or derivatives of the disclosed agents and agent classes without exceeding the scope of this disclosure.

Depending on the structure of an active agent, it may be directly coupled to an alpha-amino or an alpha-carboxylic group of a co-principle monomer in the copolymer or coupled by a linker structure to the copolymer. Such linker may function as a simple spacer between active agent and copolymer, function as a modifier of the copolymer pharmacokinetics or contain an element enabling or facilitating release of the active agent in a target cell. Linkers should be stable during storage and later in the blood stream to avoid unintended release of active agent. Release of active agent from the copolymer should take place only inside the target cells. Useful linkers (focusing on cancer therapy) should therefore be sensitive to intercellular factors such as caspases or cathepsins, glucuronidase (GUSB) (β-glucuronide-based linkers), acidic pH (found in tumor tissues or cell organelles [lysosomes]), or a reducing environment (responding to increase concentrations of intercellular glutathione). Another possibility will be the use of non-degradable linkers of the diamine type or thioether type which are not targets of a specific enzyme and are only degraded in the harsh conditions of the lysosome or peroxisomes. The latter linker type is preferred since it is associated with maximal serum stability and reduced unspecific toxicity.

In other embodiments, a copolymer is not functionalized with active agent or active agent-linker complex after synthesis but is directly synthesized as an active agent-containing copolymer by means of incorporation of co-principal monomers of formulae II-X. Active agent load is defined by the molar amounts of principal monomers, co-principle monomers of formulae III-X and co-principle monomers of formulae I and II present during polymerization. This approach is especially useful for the design of copolymers comprising combinations of different active agents as it allows active agents to be brought in both during synthesis as well as subsequent to synthesis by functionalization of co-principle monomers of formulae I and II. When the active agent is a radioisotope of short half-life, e.g., idione-123, the binding to co-principle monomers of formula IX and X (if J is H) may be performed after polymerization.

As also discussed above, copolymers containing multiple active agents can be further functionalized with cell type- or tissue type-specific targeting moieties. While this functionalization step is typically performed after active agents have been coupled to the copolymer, in special situations, e.g., in the case of active agents with a short half live such as certain radioisotopes, it may be necessary to first prepare a conjugate of a copolymer (comprising co-principle monomers of formulae I, II, IX and/or X) and a targeting moiety. Loading of the copolymer which active agents can then occur shortly prior to administration to a subject. Potential targeting moieties are, but are not limited to, monoclonal antibodies including immune checkpoint inhibitors, antibody fragments, nano-bodies (single-domain-antibodies), DARPins, peptide hormones, non-antibody proteins capable of binding to cell surface receptors, DNA/RNA-based aptamers as well as small molecules capable of binding to cell surface receptors (e.g., folic acid or biotin in the tumor context). The covalent attachment of the targeting moiety to the copolymer should be carried out in a site-specific manner to obtain a homogeneous product as well as to preserve the targeting moiety's binding affinity. Suitable coupling strategies presented in this disclosure are enzyme-catalyzed reactions with peptides tags, e.g., sortase-mediated coupling, aldehyde tags, or transglutaminase tags, or the so-called “click” reaction between copolymer and targeting moiety. The latter process may be achieved through integration during synthesis of reactive, non-canonical (unnatural) amino acids into a proteinaceous targeting moiety, e.g., an antibody (such as by means of a codon expansion technique that uses a reprogrammed stop codon that is recognized by a tRNA for an unnatural amino acid). Among the above-mentioned methods, sortase-mediated coupling is a preferred method for the site-directed coupling of a copolymer to a targeting moiety. Sortase refers to a group of prokaryotic enzymes that modify surface proteins by recognizing and cleaving a carboxyl-terminal sorting signal. For Staphylococcus aureus-derived enzymes the recognition signal consists of the motif LPXTG (Leu-Pro-any-Thr-Gy) and for Staphylococcus pyogenes-derived enzymes it is LPXTA (Leu-Pro-any-Thr-Ala). The signal sequence is preceded by a highly hydrophobic transmembrane sequence and a cluster of basic residues such as arginine. Cleavage occurs between the Thr and Gly/Ala residues of the signal sequence, with transient attachment of the Thr residue to the active site Cys residue of the sortase, followed by transpeptidation that attaches the protein covalently to a cell wall component (e.g., the peptido-glycan layer of gram-positive bacteria). Cozzi, R. et al. (2011) FASEB J 25(6): 1874-86. This enzymatic mechanism can be adapted to achieve fusion of peptides or proteins and has been used recently for the preparation of ADCs. European patent appl. no. 20130159 484 (EP 2 777 714); Beerli, R R et al. (2015) PloS One 10(7): e0131177. In the approach disclosed, a monoclonal antibody was genetically modified to contain sortase motifs at the C-termini of its heavy and light chains, and a cytotoxic drug was modified to contain an oligo-glycine stretch. The sortase-catalyzed reaction added the modified drug molecules to the C-termini of the antibody chains with high efficiency, resulting in a homogeneous ADC.

By modification of the head group of a copolymer of this disclosure with an oligo-glycine stretch, the copolymer itself becomes a target for sortase-catalyzed reactions. Since the copolymer can be loaded with a multitude of active agents, this approach results in ADCs in which many active agent molecules are linked to a small number of defined (innocuous) sites in an antibody (2-4 C-terminal sortase tags per antibody molecule). Consequently, the DAR is elevated, and with it the potency of the ADC. The oligo-glycine stretch of the copolymer can be introduced at the start of the polymerization using a newly developed RAFT agent containing 2-8 glycine residues. When this functionalized RAFT agent is used, only one sortase motif is present in each copolymer molecule.

Another preferred enzymatic coupling method utilizes a transglutaminase-catalyzed reaction. Transglutaminases, also called protein-glutamine gamma-glutamyltransferases usually cross-link proteins by transferring the γ-carboxyamide group of the glutamine residue of one protein to the ε-amino group of the lysine residue of the same or another protein. Over the last two decades, these enzymes were used in diverse areas like the food industry as “meat-glue” (Martins I M et al. (2014), Appl. Microbiol. Biotechnol. 98: 6957-64?), tissue engineering (Ehrbar M. et al. (2007) Bio-macromolecules, 8(10):3000-7), modification of therapeutic proteins (Mero A. et al. (2011) J Control Release, 154(1):27-34) or gene delivery (Trentin D. et al. (2005) J Control Release, 102(1):263-75).

In this context, microbial transglutaminases (MTgs) are the preferred class of enzymes as they are, in contrast to endogenous human transglutaminases, calcium- and nucleotide-independent enzymes. They consist of a single domain, compared to the four domains of human transglutaminases, and have about half the molecular weight of human transglutaminases. Moreover, MTgs operate at a larger range of pH values, buffers, and temperatures and have a much larger list of potential substrates. Kieliszek M et al. (2014) Rev Folia Microbiol. 59: 241-50; Martins I M. et al. (2014).

In analogy to the sortase-mediated coupling strategy, a transglutaminase motif is introduced to the head group of a copolymer of this disclosure by modification of a RAFT-agent, ensuring that only one transglutaminase motif is introduced per polymer chain. Suitable motifs are small peptides such as, but not limited to, FKGG (Ehrbar M. et al. (2007)) as potential lysine acceptor sequence, and LQSP or TQGA (Caporale A. et al. (2015) Biotechnol J. 10(1):154-61) as glutamine acceptor sequences [in this case an reactive lysine residue in the cancer cell specific targeting moiety is used]; or a monodisperse PEG spacer of 5-25 units' length containing a terminal amino group as potential glutamine acceptor sequences. From an economical perspective, amino-PEG spacers are the most preferred motifs for copolymers of this disclosure as they can be introduced without solid phase synthesis and complex protection strategies.

A variant of this strategy utilizes the transglutaminase for site-directed attachment of a click-reactive group (e.g. an azide or a tetrazine) to the targeting moiety, e.g. a monoclonal antibody, which antibody-linked reactive group is subsequently used for reaction with an “opposite” click-reactive group (alkyne or/strained alkene) at the polymeric head group of a copolymer of this disclosure. The mentioned reactive parts at the copolymer/antibody are meant to be interchangeable.

Other methods for linking a targeting moiety to a copolymer may be employed. Targeting antibodies or other polypeptides may be altered post-translationally, e.g., by converting a hydroxyl function in an amino acid side chain to a reactive aldehyde. In the case of polynucleotide-based targeting moieties, e.g., aptamers, coupling to a copolymer of this disclosure might be achieved by reaction with reactive functional groups (for instance amines, thiols, aldehydes) integrated into the aptamer during solid phase synthesis. Other site-directed coupling techniques that are well known in the art can be used to couple a copolymer to a targeting moiety.

### Pharmaceutical Compositions

The pharmaceutical compositions of the present disclosure comprise an effective amount of an active moiety of the present disclosure formulated together with one or more pharmaceutically acceptable carriers or excipients.

The pharmaceutical compositions of this disclosure may be administered parenterally, by inhalation spray, topically, rectally, nasally, buccally, vaginally or via an implanted reservoir, preferably by administration by injection (or infusion). The pharmaceutical compositions of this disclosure may contain any conventional non-toxic pharmaceutically acceptable carrier, adjuvant or vehicle. In some cases, the pH of the formulation may be adjusted with pharmaceutically acceptable acids, bases or buffers to enhance the stability of the formulated active moiety or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent. Among the acceptable vehicles and solvents that may be employed are water, Ringer's solution, U.S.P. and isotonic sodium chloride solution. Solubilizing excipients include water-soluble organic solvents such as polyethylene glycol 300, polyethylene glycol 400, ethanol, propylene glycol, glycerin, N-methyl-2-pyrrolidone, dimethylacetamide and dimethylsulfoxide; non-ionic surfactants such as Cremophor EL, Cremophor RH40, Cremophor RH60, Solutol HS15, d-α-tocopherol polyethylene glycol 1000 succinate, polysorbate 20, polysorbate 80, sorbitan monooleate, poloxamer 407, Labrafil M-1944CS, Labrafil M-2125CS, Labrasol, Gellucire 44/14, Softigen 767, and mono- and di-fatty acid esters of PEG 300, 400 and 1750; water-insoluble lipids such as castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil, various cyclodextrins such as α-cyclodextrin, β-cyclodextrin, hydroxypropyl-β-cyclodextrin (e.g., Kleptose), and sulfobutylether-β-cyclodextrin (e.g., Captisol); and phospholipids such as lecithin, hydrogenated soy phosphatidylcholine, distearoylphosphatidylglycerol, L-α-dimyristoylphosphatidylcholine and L-α-dimyristoyl-phosphatidylglycerol. Strickley (2004) Pharm. Res. 21: 201-30.

The injectable formulations can be sterilized, for example, by filtration through a bacteria-retaining filter, or by incorporating sterilizing agents in a sterile solid composition (or sterilize the solid composition by irradiation) which subsequently can be dissolved or dispersed in sterile water or other sterile injectable medium prior to use.

In order to prolong the effect of an active agent, it is often desirable to slow the absorption of an active agent from subcutaneous or intramuscular injection. Delayed absorption of a parenterally administered active moiety is accomplished by dissolving or suspending the active moiety in an oil vehicle. Injectable depot forms are made by microencapsulating the active moiety in biodegradable polymers such as polylactide-polyglycolide. Depending upon the ratio of active moiety to polymer and the nature of the particular polymer employed, the rate of active agent release can be controlled. Examples of other biodegradable polymers include poly(orthoesters) and poly(anhydrides). Depot injectable formulations are also prepared by entrapping the active moiety in liposomes or microemulsions that are compatible with body tissues.

Compositions for rectal or vaginal administration are preferably suppositories which can be prepared by mixing an active moiety of this disclosure with a suitable non-irritating excipient or carrier such as cocoa butter, polyethylene glycol or a suppository wax which excipients/carriers are solid at ambient temperature but liquid at body temperature and therefore melt in the rectum or the vaginal cavity and release the active moiety (and, consequently, the active agent).

Dosage forms for topical or transdermal administration of an active moiety of this disclosure include ointments, pastes, creams, lotions, gels, powders, solutions, sprays, inhalants or patches. The active moiety is admixed under sterile conditions with a pharmaceutically acceptable carrier and any preservatives or buffers as may be required. Ophthalmic formulations, ear drops, eye ointments, powders and solutions are also contemplated as being within the scope of this disclosure.

The ointments, pastes, creams and gels may contain, in addition to an active moiety of this disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active moiety of this disclosure, excipients such as lactose, talc, silicic acid, aluminum hydroxide, calcium silicates and polyamide powder, or mixtures of these substances. Sprays can additionally contain customary propellants.

Transdermal patches can be made by dissolving or dispensing the active moiety in the proper medium. Absorption enhancers can also be used to increase the flux of the active moiety across the skin. The rate can be controlled by either providing a rate-controlling membrane or by dispersing the active moiety in a polymer matrix or gel.

For pulmonary delivery, a pharmaceutical composition of the disclosure is formulated and administered to the patient in solid or liquid particulate form by direct administration e.g., inhalation into the respiratory system. Solid or liquid particulate forms of the active moiety prepared for practicing the present disclosure include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. Nos. 5,767,068, 5,508,269 and WO 98/43650). A discussion of pulmonary delivery of antibiotics is also found in U.S. Pat. No. 6,014,969.

The total daily dose of an active moiety of this disclosure administered to a human subject or patient in single dose or in divided doses preferably includes 0.01 to 50 mg/kg body weight of active agent or, more preferably, 0.1 to 30 mg/kg body weight of active agent. Single dose compositions may contain such amounts or submultiples thereof to make up the daily dose. In general, treatment regimens according to the present disclosure comprise administration to a human subject in need of such treatment from about 1 mg to about 5000 mg of active agent (comprised in an active moiety of this disclosure) per day in single dose or divided doses. Doses for mammalian animals can be estimated based on the latter human doses.

An active moiety of this disclosure can be administered, for example, by injection, intravenously, intraarterially, subdermally, intraperitoneally, intramuscularly, or subcutaneously; or buccally, nasally, transmucosally, topically, in an ophthalmic preparation, or by inhalation, as a daily dose comprising about 0.01 to about 50 mg/kg of body weight of active agent. Alternatively, dosages (based on a daily dose of between about 1 mg and 5000 mg active agent) may be administered every 4 to 120 hours, or according to the requirements of the particular active moiety. The methods herein contemplate administration of an effective amount of an active moiety (in a pharmaceutical composition) to achieve the desired or stated effect. Typically, the pharmaceutical compositions of this disclosure will be administered from about 1 to about 6 times per day or alternatively, as a continuous infusion. Such administration can be used as a chronic or acute therapy. The amount of active moiety that may be combined with pharmaceutically acceptable excipients or carriers to produce a single dosage form will vary depending upon the host treated and the particular mode of administration. A typical composition will contain from about 5% to about 95% active moiety (w/w). Alternatively, such preparations may contain from about 20% to about 80% active active moiety. Specific dosage and treatment regimens for any particular patient will depend upon a variety of factors, including the activity of the specific active moiety employed, the age, body weight, general health status, sex, diet, time of administration, rate of excretion, drug combination, the severity and course of the disease, condition or symptoms, the patient's disposition to the disease, condition or symptoms, and the judgment of the treating physician.

All references cited in this application, including publications, patents and patent applications, shall be considered as having been incorporated in their entirety.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or embodiment of the invention using terms such as reference to an element or elements is intended to provide support for a similar aspect or embodiment of the disclosure that “consists of’,” “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

The present disclosure, thus generally described, will be understood more readily by reference to the following examples, which are provided by way of illustration and are not intended to be limiting of the present invention.

### EXAMPLES

Note: In the examples relating to synthesis of side chain-linked amino acids names are first given in IUPAC nomenclature. Thereafter, abbreviated names are used. Table 1 shows the correspondence.

TABLE 1
IUPAC names and abbreviations

| IUPAC | Abbreviation |
|---|---|
| Co-principal monomers | |
| (S)-6-acrylamido-2-aminohexanoic acid | Acryloyl-L-lysine, AK |
| (2S)-3-(acryloyloxy)-2-aminopropanoic acid | Acryloyl-L-serine, AS |
| (2S)-3-(acryloyloxy)-2-aminobutanoic acid | Acryloyl-L-threonine, AT |
| (S)-3-(4-(acryloyloxy)phenyl)-2-aminopropanoic acid | Acryloyl-L-tyrosine, AY |
| (S)-2-(4-acrylamidophenyl)-2-aminoacetic acid | Acryloyl-L-amino- |
| | phenylalanine, AHOX |
| (2S)-4-(acryloyloxy)pyrrolidine-2-carboxylic acid | Acryloyl-L-cysteine, AC |
| (R)-3-(acryloylthio)-2-aminopropanoic acid | Acryloyl-L-oxi-proline, |
| | AHOP |
| (R)-5(3-(5-acrylamido-1-carboxypentyl)thioureido)-2-(6-hydroxy- | AK-Fluorescein-V1 |
| 3-oxo-3H-xanthen-9yl)benzoic acid | |
| (R)-5-((5-acrylamido-1-carboxypentyl)carbonyl-2-(6-hydroxy-3- | AK-Fluorescein-V2 |
| oxo-3H-xanthen-9yl)benzoic acid | |
| (S)-6-acrylamido-2-(4-(((2S,3S,4S,6R)-3-hydroxy-2-methyl-6- | AK-DOX-V1 |
| (((1S,3S)-3,5,12-trihydroxy-3-(2-hydroxyacetyl)-10-methoxy- | |
| 6,11-dioxo-1,2,3,4,6,11-hexahydrotetracen-1-yl)oxy)tetrahydro- | |
| 2H-pyran-4-yl)amino)-4-oxobutanamido)hexanoic acid | |
| (2S)-6-acrylamido-2-((1-(6-(((S)-1-(((S)-1-((4-(((((2S,3S,4S,6R)- | AK-DOX-V2 |
| 3-hydroxy-2-methyl-6-(((1S,3S)-3,5,12-trihydroxy-3-(2- | |
| hydroxyacetyl)-10-methoxy-6,11-dioxo-1,2,3,4,6,11- | |
| hexahydrotetracen-1-yl)oxy)tetrahydro-2H-pyran-4- | |
| yl)carbamoyl)oxy)methyl)phenyl)amino)-1-oxo-5-ureidopentan-2- | |
| yl)amino)-3-methyl-1-oxobutan-2-yl)amino)-6-oxohexyl)-2,5- | |
| dioxopyrrolidin-3-yl)amino)hexanoic acid | |
| (S)-6-acrylamido-2-(3-(4-hydroxyphenyl)propanamido)hexanoic | AK-Phenol |
| acid | |
| (S)-2,2′,2″-(10-(2-((5-acrylamido-1-carboxypentyl)amino)-2- | AK-DOTA |
| oxoethyl)-1,4,7,10-tetraazacyclododecane-1,4,7-triyl)triacetic | |
| acid | |
| Polymeric substances | |
| α-ethyl-trithiocarbonate-ω-tert-butyl (14-methyl-2,5,8,13- | BOC-G₃-DMA-RAFT pre- |
| tetraoxo-3,6,9,12-tetraazapentadecyl)carbamate poly(N,N- | polymer |
| dimethylacrylamide) | |
| α-ethyl-trithiocarbonate-ω-N-(2-(2-(2-(2- | G₃-DMA-RAFT pre- |
| aminoacetamido)acetamido)acetamido)ethyl)isobutyramide | polymer |
| poly(N,N-dimethylacrylamide) | |
| ω-tert-butyl (14-methyl-2,5,8,13-tetraoxo-3,6,9,12- | BOC-G₃-Cellophil |
| tetraazapentadecyl)carbamate poly(N,N-dimethylacrylamide-co- | |
| (S)-6-acrylamido-2-aminohexanoic acid) | |
| ω-N-(2-(2-(2-(2- | G₃-Cellophil |
| aminoacetamido)acetamido)acetamido)ethyl)isobutyramide | |
| poly(N,N-dimethylacrylamide-co-(S)-6-acrylamido-2- | |
| aminohexanoic acid) | |
| ω-tert-butyl (47-methyl-2,5,8,46-tetraoxo- | BOC-G₃-PEG₁₁-Cellophil |
| 12,15,18,21,24,27,30,33,36,39,42-undecaoxa-3,6,9,45- | |
| tetraazaoctatetracontyl)carbamate poly(N,N-dimethylacrylamide- | |
| co-(S)-6-acrylamido-2-aminohexanoic acid) | |
| ω-tert-butyl (14-methyl-2,5,8,13-tetraoxo-3,6,9,12- | BOC-G₃-Cellophil- |
| tetraazapentadecyl)carbamate poly(N,N-dimethylacrylamide-co- | (Fluorescein)₈ |
| (S)-5-(3-(4-acrylamido-1-carboxybutyl)thioureido)-2-(6-hydroxy- | |
| 3-oxo-3H-xanthen-9-yl)benzoic acid) | |
| ω-tert-butyl (47-methyl-2,5,8,46-tetraoxo- | H₂N-G₃-PEG₁₁-Cellophil- |
| 12,15,18,21,24,27,30,33,36,39,42-undecaoxa-3,6,9,45- | (Fluorescein)₈ |
| tetraazaoctatetracontyl)carbamate poly(N,N-dimethylacrylamide- | |
| co-(S)-5-(3-(4-acrylamido-1-carboxybutyl)thioureido)-2-(6- | |
| hydroxy-3-oxo-3H-xanthen-9-yl)benzoic acid) | |
| α-ethyl-trithiocarbonate-ω-N-(3-azidopropyl)isobutyramide | RAFT-DMA-N₃ pre |
| poly(N,N-dimethylacrylamide) | polymer |
| ω-N-(3-azidopropyl)isobutyramide poly(N,N-dimethylacrylamide- | Cellophil-N₃ |
| co-(S)-6-acrylamido-2-aminohexanoic acid) | |
| ω-N-(3-azidopropyl)isobutyramide poly(N,N-dimethylacrylamide- | Cellophil-(Fluorescein)₈-N₃ |
| co-(R)-5-((5-acrylamido-1-carboxypentyl)carbonyl-2-(6-hydroxy- | |
| 3-oxo-3H-xanthen-9yl)benzoic acid) | |
| α-ethyl-trithiocarbonate-ω-tert-butyl 2-((2- | RAFT-DMA-Oxime-BOC |
| isobutyramidoethyl)amino)-2-oxoethoxycarbamate-poly(N,N- | pre polymer |
| dimethylacrylamide) | |
| ω-tert-butyl 2-((2-isobutyramidoethyl)amino)-2- | Cellophil-Oxime-BOC |
| oxoethoxycarbamate-poly(N,N-dimethylacrylamide-co-(S)-6- | |
| acrylamido-2-aminohexanoic acid) | |
| ω-tert-butyl 2-((2-isobutyramidoethyl)amino)-2- | Cellophil-(Fluorescein)₈- |
| oxoethoxycarbamate-poly(N,N-dimethylacrylamide-co-(S)-5-(3- | Oxime-BOC |
| (4-acrylamido-1-carboxybutyl)thioureido)-2-(6-hydroxy-3-oxo- | |
| 3H-xanthen-9-yl)benzoic acid) | |
| ω-N-(2-(2-(aminooxy)acetamido)ethyl)isobutyramide-poly(N,N- | Cellophil-(Fluorescein)₈- |
| dimethylacrylamide-co-(S)-5-(3-(4-acrylamido-1- | Oxime |
| carboxybutyl)thioureido)-2-(6-hydroxy-3-oxo-3H-xanthen-9- | |
| yl)benzoic acid) | |
| α-ethyl-trithiocarbonate-ω-isobutyric acid-poly(N,N- | RAFT-Cellophil-CO₂H |
| dimethylacrylamide-co-(S)-6-acrylamido-2-aminohexanoic acid) | |
| tert-butyl(20-methyl-19-oxo-3,6,9,12,15-pentaoxa-18- | Cellophil BOC-NH-PEG₅- |
| azahenicosyl)carbamate-poly(N,N-dimethylacrylamide-co-(S)-6- | (DMA₃₀/AK₈) |
| acrylamido-2-aminohexanoic acid) | |
| N-(17-amino-3,6,9,12,15-pentaoxaheptadecyl)isobutyramide- | Cellophil NH₂-PEG₅- |
| poly(N,N-dimethylacrylamide-co-(S)-2,2′,2″-(10-(2-((5- | (DMA₃₀/AK-DOTA₈) |
| acrylamido-1-carboxypentyl)amino)-2-oxoethyl)-1,4,7,10- | |
| tetraazacyclododecane-1,4,7-triyl)triacetic acid) | |
| N¹-dibenzocylcooctyne-N⁶-(20-methyl-19-oxo-3,6,9,12,15- | Cellophil DBCO-NH- |
| pentaoxa-18-azahenicosyl)adipamide poly(N,N- | PEG5-(DMA₃₀/AK-DOTA₈) |
| dimethylacrylamide-co-(S)-2,2′,2″-(10-(2-((5-acrylamido-1- | |
| carboxypentyl)amino)-2-oxoethyl)-1,4,7,10- | |
| tetraazacyclododecane-1,4,7-thiyl)triacetic acid) | |
| α-cycloalkyne-ω-isobutyric acid-poly(N,N-dimethylacrylamide- | Cycloalkyne- |
| co-(S)-2,2′,2″-(10-(2-((5-acrylamido-1-carboxypentyl)amino)-2- | Cellophil(DOTA)-CO₂H |
| oxoethyl)-1,4,7,10-tetraazacyclododecane-1,4,7-triyl)triacetic | |
| acid) | |
| N-(1-(4-(6-methyl-1,2,4,5-tetrazin-3-yl)phenyl)-2-oxo- | Tetrazine-Cellophil |
| 6,9,12,15,18-pentaoxa-3-azaicosan-20-yl)isobutyramide | [DMA₃₀/AK-DOTA₈]. |
| poly(N,N-dimethylacrylamide-co-(S)-2,2′,2″-(10-(2-((5- | |
| acrylamido-1-carboxypentypamino)-2-oxoethyl)-1,4,7,10- | |
| tetraazacyclododecane-1,4,7-triyl)triacetic acid) | |
| 6-amino-9-(2-carboxy-5-((5-(((((4aS,7R)-4-methyl-1-(4-(23- | AFDye-488-Click-Cellophil |
| methyl-2,22-dioxo-6,9,12,15,18-pentaoxa-3,21- | [DMA₃₀/AK-DOTA₈] |
| diazatetracosyl)phenyl)-2,4a,5,6,7,8,9,10- | |
| octahydrocycloocta[d]pyridazin-7- | |
| yl)oxy)carbonyl)amino)pentyl)carbamoyl)phenyl)-4,5-disulfo-3H- | |
| xanthen-3-iminium poly(N,N-dimethylacrylamide-co-(S)-2,2′,2″- | |
| (10-(2-((5-acrylamido-1-carboxypentypamino)-2-oxoethyl)- | |
| 1,4,7,10-tetraazacyclododecane-1,4,7-triyl)triacetic acid) | |
| Intermediates | |
| 2-(((ethylthio)carbonothioyl)thio)-2-methylpropanoic acid | Ethyl-RAFT |
| 2,5-dioxopyrrolidin-1-yl 2-(((ethylthio)carbonothioyl)thio)-2- | RAFT-NHS |
| methylpropanoate | |
| tert-butyl (2-(2-(((ethylthio)carbonothioyl)thio)-2- | RAFT-EDA-BOC |
| methylpropanamido)ethyl)carbamate | |
| 2-(2-(((ethylthio)carbonothioyl)thio)-2- | RAFT-EDA-OTf |
| methylpropanamido)ethanaminium 2,2,2-trifluoroacetate | |
| tert-butyl (6,6-dimethyl-7,12,15,18-tetraoxo-4-thioxo-3,5-dithia- | BOC-G₃-RAFT |
| 8,11,14,17-tetraazanonadecan-19-yl)carbamate | |
| (S)-6-(((allyloxy)carbonyl)amino)-2-aminohexanoic acid | H-Lys(Alloc)-OH |
| (S)-2,5-dioxopyrrolidin-1-yl 2-((((9H-fluoren-9- | FMOC-Val-OSu |
| yl)methoxy)carbonyl)amino)-3-methylbutanoate | |
| (S)-2-((S)-2-((((9H-fluoren-9-yl)methoxy)carbonyl)amino)-3- | FMOC-Val-Lys(Alloc)-OH |
| methylbutanamido)-6-(((allyloxy)carbonyl)amino)hexanoic acid | |
| (9H-fluoren-9-yl)methyl ((S)-1-(((S)-6- | FMOC-Val-Lys(Alloc)- |
| (((allyloxy)carbonyl)amino)-1-((4-(hydroxymethyl)phenyl)amino)- | PABOH |
| 1-oxohexan-2-yl)amino)-3-methyl-1-oxobutan-2-yl)carbamate | |
| allyl ((S)-5-((S)-2-amino-3-methylbutanamido)-6-((4- | H-Val-Lys(Alloc)-PABOH |
| (hydroxymethyl)phenyl)amino)-6-oxohexyl)carbamate | |
| allyl ((S)-5-((S)-2-(6-(2,5-dioxo-2,5-dihydro-1H-pyrrol-1- | MC-Val-Lys(Alloc)-PABOH |
| yl)hexanamido)-3-methylbutanamido)-6-((4- | |
| (hydroxymethyl)phenyl)amino)-6-oxohexyl)carbamate | |
| allyl ((S)-5-((S)-2-(6-(2,5-dioxo-2,5-dihydro-1H-pyrrol-1- | MC-Val-Lys(Alloc)-PABO- |
| yl)hexanamido)-3-methylbutanamido)-6-((4-((((4- | PNP |
| nitrophenoxy)carbonyl)oxy)methyl)phenyl)amino)-6- | |
| oxohexyl)carbamate | |
| allyl ((S)-5-((S)-2-(6-(2,5-dioxo-2,5-dihydro-1H-pyrrol-1- | MC-Val-Lys(Alloc)-PABC- |
| yl)hexanamido)-3-methylbutanamido)-6-((4-(((((2S,3S,4S,6R)-3- | DOX |
| hydroxy-2-methyl-6-(((1S,3S)-3,5,12-trihydroxy-3-(2- | |
| hydroxyacetyl)-10-methoxy-6,11-dioxo-1,2,3,4,6,11- | |
| hexahydrotetracen-1-yl)oxy)tetrahydro-2H-pyran-4- | |
| yl)carbamoyl)oxy)methyl)phenyl)amino)-6-oxohexyl)carbamate | |
| 4-((S)-6-amino-2-((S)-2-(6-(2,5-dioxo-2,5-dihydro-1H-pyrrol-1- | MC-Val-Lys-PABC-DOX |
| yl)hexanamido)-3-methylbutanamido)hexanamido)benzyl | |
| ((2S,3S,4S,6R)-3-hydroxy-2-methyl-6-(((1S,3S)-3,5,12- | |
| trihydroxy-3-(2-hydroxyacetyl)-10-methoxy-6,11-dioxo- | |
| 1,2,3,4,6,11-hexahydrotetracen-1-yl)oxy)tetrahydro-2H-pyran-4- | |
| yl)carbamate | |
| 4-((S)-6-amino-2-((S)-2-(6-(2,5-dioxo-2,5-dihydro-1H-pyrrol-1- | MC-Ala-Lys-PABC-DOX |
| yl)hexanamido)propanamido)hexanamido)benzyl | |
| ((2S,3S,4S,6R)-3-hydroxy-2-methyl-6-(((1S,3S)-3,5,12- | |
| trihydroxy-3-(2-hydroxyacetyl)-10-methoxy-6,11-dioxo- | |
| 1,2,3,4,6,11-hexahydrotetracen-1-yl)oxy)tetrahydro-2H-pyran-4- | |
| yl)carbamate | |
| 4-((S)-2-((S)-2-(6-(2,5-dioxo-2,5-dihydro-1H-pyrrol-1- | MC-Val-Cit-PABO-PNP |
| yl)hexanamido)-3-methylbutanamido)-5- | |
| ureidopentanamido)benzyl (4-nitrophenyl) carbonate | |
| 2,2′,2″-(10-(2-((2,5-dioxopyrrolidin-1-yl)oxy)-2-oxoethyl)- | DOTA-NHS |
| 1,4,7,10-tetraazacyclododecane-1,4,7-triyl)triacetic acid | |
| 2,2′,2″-(10-(2,6-dioxotetrahydro-2H-pyran-3-yl)-1,4,7,10- | DOTA-Anhydride |
| tetraazacyclododecane-1,4,7-triyl)triacetic acid | |
| 2,2′,2″-(10-(1-carboxy-4-((4-isothiocyanatobenzyl)amino)-4- | p-NCS-Bz-DOTA-GA |
| oxobutyl)-1,4,7,10-tetraazacyclododecane-1,4,7-triyl)triacetic | |
| acid | |
| 4-((S)-2-((S)-2-(6-(2,5-dioxo-2,5-dihydro-1H-pyrrol-1- | MC-Val-Cit-PABC-DOX |
| yl)hexanamido)-3-methylbutanamido)-5- | |
| ureidopentanamido)benzyl ((2S,3S,4S,6R)-3-hydroxy-2-methyl- | |
| 6-(((1S,3S)-3,5,12-trihydroxy-3-(2-hydroxyacetyl)-10-methoxy- | |
| 6,11-dioxo-1,2,3,4,6,11-hexahydrotetracen-1-yl)oxy)tetrahydro- | |
| 2H-pyran-4-yl)carbamate | |
| Chemicals | |
| N-Hydroxysuccinimide | NHS |
| N-Ethyl-N′-(3-dimethylaminopropyl)carbodiimide hydrochloride | EDC•HCl |
| 2,2′-Azobis[2-(2-imidazolin-2-yl)propane]dihydrochloride | VA044 |
| 4-aminobenzyl alcohol | PABOH |
| N-Ethoxycarbonyl-2-ethoxy-1,2-dihydroquinoline | EEDQ |
| Diisopropylethylamine | DIEA |
| 4-Nitrophenyl chloroformate | PNP chloroformate |
| N-methyl-2-pyrrolidone | NMP |
| Dichloromethane | DCM |
| Tetrahydrofuran | THF |
| Ethyl acetate | EtOAc |
| Methanol | MeOH |
| N-alpha-(9-Fluorenylmethyloxycarbonyl)-L-valine succinimidyl | FMOC-Val-OSu |
| ester | |

### Example 1: Synthesis of (S)-6-Acrylamido-2-Aminohexanoic Acid Monomers Via Copper Complex

L-lysine (14.62 g; 100 mmol) was dissolved in 150 mL deionized water and heated to about 80° C. Copper carbonate (16.6 g; 75 mmol) was added in portions over a period of 30 minutes. The reaction was stirred for an additional 30 minutes. The hot, deep-blue suspension was filtered through silica gel. The filter was washed with a small amount of water. On the subsequent day, the lysine copper complex-containing the combined filtrate was cooled in an ice bath, and 100 mL tetrahydrofuran (THF) were added. A solution of acryloyl chloride in methyl-tert-butylether (TBME) (8.9 mL, 110 mmol) was added dropwise during a period of one hour. The pH was initially maintained between 8 and 10 by parallel, dropwise addition of 10% sodium hydroxide solution. After half the acryloyl chloride solution had been added, the product began to precipitate. When most of the acryloyl chloride had been added, the addition of sodium hydroxide was slowed down to allow the pH to drop to about 6 and the temperature of the reaction mixture to reach room temperature. The blue suspension was stirred during an additional 2 hours and was then filtered. The solid material retained on the filter was washed with water and acetone and then dried. A yield of 6.5 g of acryloyl-L-lysine copper complex was obtained.

Acryloyl-L-lysine copper complex (29.5 g) was suspended in 300 mL deionized water and cooled in an ice bath. H₂S gas was bubbled into the suspension until the copper sulfide precipitation was complete. Three grams of active charcoal were added to the suspension. The suspension was heated briefly to 100° C. After cooling to room temperature, 500 mL acetone were added to the suspension which was then filtered on silica gel. The clear filtrate was put in a rotary evaporator. After evaporation of the solvent, the solid product was recrystallized from 200 mL of 50% aqueous acetone. A yield of 17.76 g (70%) of white powder was obtained. The structure of the compound was verified by NMR and LC-MS spectroscopy.

### Example 2: Synthesis (2S)-3-(acryloyloxy)-2-aminopropanoic Acid

A solution of L-serine (5 g, 47.6 mmol) in water (50 mL) was heated to 80° C., and solid copper carbonate (5.79 g, 26.2 mmol) was added. The solution was stirred for 10 min. Undissolved residue was subsequently collected by filtration and washed with water (30 mL). The combined filtrate was cooled in an ice bath, and KOH (27.1 mL, 47.6 mmol) was added slowly. To this solution a mixture of acryloyl chloride (4.52 mL, 59.5 mmol) in acetone (30 mL) was added dropwise. The reaction mixture was then incubated at 4° C. overnight under stirring. The formed solid was isolated and washed with water (50 mL)/methanol (50 mL)/ethyl-tert-butylether (50 mL) (MTBE) and finally dried under reduced pressure to give 0-acryloyl-L-serine-Cu²⁺ complex (3.8 g, 10.01 mmol; 42.1% yield). The copper in the complex was subsequently removed by a similar procedure as that described in example 1. A yield of 1.43 g (45%) of acryloyl-L-serine as white powder was obtained. The identity of the compound was verified by NMR and LC-MS spectroscopy.

### Example 3: Synthesis (2S)-3-(acryloyloxy)-2-aminobutanoic Acid

A reaction vessel with 6 mL trifluoroacetic acid (TFA) was cooled in an ice bath. Subsequently, solid L-threonine (2.00 g, 16.79 mmol) was added, and the mixture was stirred for 5 min. Trifluromethanesulfonic acid (0.18 mL, 2.0 mmol) and, subsequently, acryloyl chloride (2.5 mL, 32.9 mmol) were added, and the reaction mixture was incubated for 2 h at room temperature. After completion of the reaction, the product was precipitated with methyl-tert-butylether (MTBE). After isolation of the solid, the product was washed with MTBE and acetone. O-Acryloyl-L-threonine hydrochloride was finally dried under reduced pressure to give a white powder (yield 32%). The structure of the compound was verified by NMR and LC-MS spectroscopy.

### Example 4: Synthesis of (S)-3-(4-(acryloyloxy)phenyl)-2-aminopropanoic Acid

The synthesis of O-acryloyl-L-tyrosine-Cu²⁺-complex was performed according to the procedure described in Example 1. Copper was removed from the complex by the following procedure: 73.15 g (140 mmol) of O-acryloyl-L-tyrosine-Cu²⁺-complex was dissolved in 220 mL 2 N HCl in a grinding dish. The mixture was homogenized using Polytron® PT 3000 equipment. Subsequently, the mixture was filtered and the residue washed twice with 50 mL 2 N HCl. The solid compound was then dried over NaOH at 40° C. under reduced pressure to give O-acryloyl-L-tyrosine hydrochloride (46.96 g, 63% yield).

### Example 5: Synthesis of (S)-2-(4-acrylamidophenyl)-2-aminoacetic Acid

Boc-4-amino-L-phenylalanine (2.50 g, 8.9 mmol, Anaspec, Fremont, Calif.) was dissolved in 25 mL chloroform. Triethylamine (2.47 mL, 17.8 mmol) was given to this solution, and the mixture was cooled to −15° C. Subsequently, acryloyl chloride (0.79 mL, 9.8 mmol) in chloroform was added dropwise to the mixture under stirring. After the acryloyl chloride addition was completed, the reaction mixture was stirred for three additional hours. The reaction mixture was thereafter passed through a glass filter, the protected (S)-2-(4-acrylamidophenyl)-2-aminoacetic acid was purified by column chromatography, and the residual solvents were evaporated. The obtained (S)-2-(4-acrylamidophenyl)-2-((tert-butoxycarbonyl)amino)acetic acid (500 mg, 1.5 mmol) was dissolved in 5 mL dichloromethane (DCM). Trifluoracetic acid (TFA) (800 μL, 10.38 mmol) was added, and the solution was stirred for 1 h at room temperature. Afterwards, the solvent was removed under reduced pressure, 5 mL DCM were added, and the solvent was again removed under reduced pressure. This procedure was repeated several times. Finally, the product was dissolved in 3 mL DCM and precipitated with methyl-tert-butylether (MTBE). The solid was collected on a glass filter and dried in vacuo to obtain pure acryloyl-4-amino-L-phenylalanine at a yield of 15%. The structure of the compound was verified by NMR.

### Example 6: Synthesis of (2S)-4-(acryloyloxy)pyrrolidine-2-carboxylic Acid and (R)-3-(acryloylthio)-2-aminopropanoic Acid

The synthesis of these compounds was performed as described in Example 1. For (2S)-4-(acryloyloxy)pyrrolidine-2-carboxylic acid and (R)-3-(acryloylthio)-2-aminopropanoic acid, the starting materials were, respectively, 4-hydroxy-L-proline and L-cysteine.

### Example 7: Synthesis of (S)-6-acrylamido-2-(3-(4-hydroxyphenyl)propanamido) Hexanoic Acid (AK-Phenol)

To a solution of AK (538 mg, 2.69 mmol) and TEA (443 μL, 3.18 mmol) in DMF (9 mL) was added Bolton-Hunter Reagent (643 mg, 2.44 mmol). The reaction mixture was stirred overnight at room temperature. The reaction mixture was then filtered, and the volatiles were removed under a stream of N₂. The residue was purified by SiO₂ column chromatography. The structure was verified by NMR spectroscopy.

### Example 8: Synthesis of Methacryl/Ethylacryl/Propylacryl Derivatives of Amino Acids

Syntheses of methacryl/ethylacryl/propylacryl-derivatives were performed using the respective acid chloride, e.g. methacryloyl chloride, under conditions described in Examples 1 to 7.

### Example 9: Synthesis of Fluorescein-Modified Co-Principal Monomer AK-Fluorescein-V1

In a 20 mL reaction vessel a solution of acryloyl-L-lysine [see example 1] (150 mg, 0.749 mmol), fluorescine isothiocyanate (FITC, 321 mg, 0.824 mmol) and triethylamine (0.114 mL, 0.824 mmol) was prepared in DMF. The reaction was incubated overnight in the dark and at room temperature under constant stirring. Subsequently, the solution was filtered through a 0.4-μm filter to remove potential particles. Afterwards, the residual solvent was removed by applying vacuum in a rotary evaporator at 30° C. Structure was confirmed by NMR and LC-MS (yield 98%, with a purity of >95%). The synthesized monomer was afterward tested in a copolymerization with dimethylacrylamide (DMA) [90/10 mol/mol] using DMF as solvent and AIBN as initiator. The polymerization reaction was carried out at 65° C. for 6 h, and the resulting copolymer was analyzed by gel permeation chromatography (GPC) using the protocol presented in example 13.

### Example 10: Synthesis of Fluorescein-Modified Co-Principal Monomer AK-Fluorescein-V2

The reaction was performed with Fluorescein-NHS as starting material according to the synthesis protocols presented in example 9 but with a 10 mol % excess of AK which was removed after reaction by precipitation.

Structure was confirmed by NMR and LC-MS (yield 85%, with a purity of >93%). The synthesized monomer was afterward tested in a copolymerization with dimethylacrylamide (DMA) [90/10 mol/mol] using DMF as solvent and AIBN as initiator. The polymerization reaction was carried out at 65° C. for 6 h, and the resulting copolymer was analyzed by GPC using the protocol presented in example 13.

### Example 11: Synthesis of a Doxorubicin (DOX)-Modified Co-Principle Monomer with a Non-Cleavable Linker (AK-DOX-V1)

To a solution of DOX-HCl (200 mg, 345 μmol, 1.00 eq.) and Et₃N (50 μL, 348 μmol, 1.01 eq.) in DMF was added succinic anhydride (36.2 mg, 362 μmol, 1.05 eq.). The mixture was stirred under inert atmosphere at room temperature for 30 min and then NHS (43.7 mg, 379 μmol, 1.10 eq.) and, subsequently, EDC-HCl (69.4 mg, 362 μmol, 1.05 eq.) were added. The resulting mixture was stirred overnight at room temperature and then AK (69.0 mg, 345 μmol, 1.00 eq.) and, subsequently, Et₃N (53 μL, 379 μmol, 1.10 eq.) were added. The reaction mixture was stirred again overnight at room temperature. The volatiles were evaporated under a stream of N₂, and the residue was purified by SiO₂ column chromatography to obtain the desired product (228 mg, 276 μmol, 80%).

### Example 12: Synthesis of a Doxorubicin Modified Co-Principle Monomer with a Cathepsin B Sensitive Linker (AK-DOX-V2)

A solution of DOX-HCl (86 mg, 149 μmol, 1.10 eq.), MC-Val-Cit-PABO-PNP (100 mg, 136 μmol, 1.00 eq.) and N,N-diisopropylethylamine (DIPEA) (26 μL, 149 μmol, 1.10 eq.) in N-methyl-2-pyrrolidone (NMP) is stirred for 2 h at room temperature. To the resulting mixture are added AK (28.5 mg, 142 μmol, 1.05 eq.) followed by DIPEA (26 μL, 149 μmol, 1.10 eq.). The reaction mixture is stirred overnight at room temperature. The volatiles are evaporated under a stream of N₂, and the residue is purified by chromatography on SiO₂ to obtain the desired product (109 mg, 81 μmol, 60%).

### Example 13: General Procedure for the Synthesis of BOC-Gₙ-Cellophil

### Step 1: Synthesis of the RAFT-NHS Intermediate

To a solution of 2-[[(Ethylthio)thioxomethyl]thio]-2-methyl-propanoic acid (22.85 g, 102 mmol, 1.0 eq.), synthesized as described in Tucker et al. (ACS Macro Letters (2017) 6(4): 452-457), and 1-hydroxypyrrolidinine-2,5-dione (12.89 g, 112 mmol, 1.1 eq.) in CH₂Cl₂ was added EDC-HCl (21.48 g, 112 mmol, 1.1 eq.) at 0° C. The reaction mixture was allowed to stir at room temperature for 16 h. The reaction mixture was then partially evaporated (to about half of the total volume) under a flow of N₂ and diluted with AcOEt and double-distilled water (ddH₂O). The biphasic solution was transferred in a separating funnel and, after extraction, the organic phase was successively washed with ddH₂O, an aqueous saturated solution of NaHCO₃ (3×), ddH₂O (2×) and brine. The organic phase was dried (Na₂SO₄), and all volatiles were removed under reduced pressure. The residue was triturated with n-hexane and the resulting yellow suspension was filtered. The cake was washed with n-hexane. The yellow solid was dried under reduced pressure, and the resulting intermediate (RAFT-NHS) was used without further purification (31.8 g, 99.0 mmol, 97%). All analytical data were in agreement with literature values. Yang et al. (2012) Macromolecular rapid communications 33(22):1921-6.

### Step 2: Synthesis of the RAFT-EDA-BOC Intermediate

To a solution of RAFT-NHS starting material (1.22 g, 3.61 mmol 1.0 eq.) in CH₂Cl₂ was added dropwise and at −10° C. a solution of t-butyl-(2-aminoethyl) carbamate (0.81 g, 5.0 mmol, 1.4 eq.) and Et₃N (1.0 mL, 7.2 mmol, 2.0 eq.) in CH₂Cl₂. The reaction mixture was stirred for 12 h at room temperature. The organic mixture was successively washed with an aqueous saturated solution of NH₄Cl (2×), an aqueous saturated solution of NaHCO₃ (2×), and brine. The organic phase was dried (Na₂SO₄), and all volatiles were removed under reduced pressure. The residue was recrystallized from a mixture of n-heptane and Et₂O. The yellow crystals were filtrated, washed with n-heptane and dried under reduced pressure to give the next intermediate (RAFT-EDA-BOC, 1.26 g, 3.44 mmol, 95%). The structure of the obtained compound was verified by MS and NMR spectroscopy.

### Step 3: Synthesis of the RAFT-EDA-OTf Intermediate

A cold solution of RAFT-EDA-BOC (1.25 g, 3.41 mmol, 1.0 eq.) in TFA was stirred for 60 min. The reaction mixture was then diluted with MeOH and CH₂Cl₂ (½), and the volatiles were partially (% of the total volume) removed under a flow of N₂. The resulting RAFT-EDA-OTf was isolated as a yellow oil (2.00 g, 3.29 mmol, 96%) and was used in the next step without further purification. The structure of the obtained compound was verified by MS and NMR spectroscopy.

### Step 4: Synthesis of a BOC-Gₙ-RAFT Intermediate

A solution of BOC-G₃ (697 mg, 2.41 mmol, 1.0 eq.), 1-hydroxybenzotriazole hydrate (HOBt hydrate) (92.0 mg, 600 μmol, 0.25 eq.) and EDC-HCl (485 mg, 2.53 mmol, 1.05 eq.) in CH₂Cl₂ was stirred for 30 min at 0° C. under inert atmosphere (N₂). To this solution was successively and dropwise added a solution of RAFT-EDA-OTf (917 mg, 2.41 mmol, 1.0 eq.) in CH₂Cl₂ and DIPEA (2.13 mL, 12.5 mmol, 5.2 eq.). The reaction mixture was stirred for 1 h at 0° C. and then overnight at room temperature. The reaction mixture was diluted with CH₂Cl₂, and the organic mixture was successively washed with a saturated solution of NH₄Cl (3×), with a saturated solution of NaHCO₃, ddH₂O and brine. The organic phase was collected, dried (Na₂SO₄), and the volatiles were partially removed (% of the total volume) under reduced pressure. To the resulting solution was added EtOAc. The resulting cloudy solution was then stored in the refrigerator overnight to obtain a yellow suspension, which was filtered, and the cake was washed with cold EtOAc. The yellow solid was dried under reduced pressure to obtain BOC-G₃-RAFT agent (396 mg, 736 μmol, 31%). The structure of the obtained compound was verified by MS and NMR spectroscopy.

### Step 5: Synthesis of a BOC-Gₙ-DMA-RAFT Pre-Polymer

To a solution of DMA (192 μL, 1.86 mmol, 10 eq.) in dioxane were successively added BOC-G₃-RAFT agent (100 mg, 186 μmol, 1.0 eq.) and AIBN (6.1 mg, 37 μmol, 0.20 eq.). The reaction mixture was stirred for 6 h at 60° C. The reaction product was then precipitated in n-hexane. The light-yellow suspension was filtered and the resulting cake washed with n-hexane and finally dissolved in acetone. The volatiles were then removed under reduced pressure to obtain BOC-G₃-DMA-RAFT pre-polymer as a yellow oil (280 mg, 186 μmol, 99%). The structure of the obtained compound was verified by MS and NMR spectroscopy.

### Step 6 (Optional): Synthesis of a Gₙ-DMA-RAFT Pre-Polymer

A solution of the BOC-G₃-DMA-RAFT pre-polymer in dioxane is treated with a solution of HCl (4 M) in dioxane for 2 h. The volatiles are removed under a flow of N₂ at room temperature. The residue is used without further purification. The structure of the obtained compound is verified by MS and NMR spectroscopy.

### Step 7: Synthesis of a BOC-Gₙ-Cellophil

To a solution of DMA (1.23 mL, 11.9 mmol, 70 eq.) and AK (272 mg, 1.36 mmol, 8 eq.) in ddH₂O were successively added BOC-G₃-DMA pre-polymer (221 mg, 170 μmol, 1.0 eq.) and VA044 (27.5 mg, 85 μmol, 0.4 eq.). The reaction mixture was stirred for 4 h at 55° C. The reaction mixture was diluted with ddH₂O and dioxane. To this solution was successively added phosphinic acid (50 w %, 93 μL, 850 μmol, 5 eq.), TEA (118 μL, 850 μmol, 5 eq.) and VA044 (27.5 mg, 85 μmol, 0.5 eq.). The reaction mixture was stirred for 4 h at 100° C. The resulting mixture was then dialyzed (MWCO 3.5 kDa) against ddH₂O and the retentate was freeze-dried to obtain BOC-G₃-Cellophil as a white powder (1.20 g, 120 μmol, 71% over two steps). The structure of the obtained compound was verified by NMR spectroscopy and GPC using the following protocol: A stock solution of 3.33 mg/mL copolymer was prepared in elution buffer (deionized water containing 0.05% (w/v) NaN₃) and filtered through a 0.45 μm syringe filter. Subsequently, 0.4 mL of stock solution was injected in the port of the GPC device (1260 Infinity LC-System, Agilent, Santa Clara, Calif.). Chromatography was performed at a constant flow rate of 0.5 mL/min in elution buffer. Copolymer samples were separated on a Suprema three-column system (pre-column, 1000 Å, 30 Å; 5 μm particle size; PSS, Mainz, Germany) which was placed in an external column oven at 55° C. Copolymers were analyzed by RI (refractive index) and UV detectors. A calibration curve (10 points) was established using a pullulan standard. Molecular weights of characterized copolymers were estimated with reference to this standard.

The synthesis of the pentaglycine derivative (BOC-G₅-Cellophil) was achieved following the above protocol with minor modifications using BOC-G₅-Na salt as starting material. BOC-G₅-Na salt was obtained from pentaglycine and synthesized according to Wang, T.-P. et al (2012) Bioconjugate Chemistry 23(12): 2417-2433. Other oligoglycine-functionalized Cellophil copolymers can be generated using a similar protocol.

### Example 14: Synthesis of BOC-Gₙ-PEGₓ-Cellophil

The synthesis of BOC-G₃-PEG₁₁-Cellophil was achieved following the above general procedures (Example 13: step 1-4 and 7). Step 2 was executed using BOC-PEG₁₁-CH₂CH₂—NH₂ as starting material to yield RAFT-PEG₁₁-BOC, and step 3 was carried out using HCl in EtOAc.

The synthesis of the PEG₂₃ derivative (BOC-G₃-PEG₂₃-Cellophil) was achieved following the above protocol with minor modifications using BOC-PEG₂₃-CH₂CH₂—NH₂ as starting material. Other oligoglycine- and PEG-functionalized Cellophil copolymers can be generated by a similar protocol.

### Example 15: Synthesis of a BOC-Gₙ-Cellophil-(Fluorescein)₈ or a BOC-Gₙ-PEGₓ-Cellophil-(Fluorescein)₈

To a solution of BOC-G₃-Cellophil (1.0 eq.) or BOC-G₃-PEG-Cellophil (1.0 eq.) (for preparation see example 13, step 7) in an aqueous solution of NaHCO₃ (0.1 N) was slowly added a solution of FITC (16 eq.) in DMSO. The resulting reaction mixture was stirred for 16 h at room temperature and dialyzed (MWCO 3.5 kDa) against an aqueous solution of NaHCO₃ (0.1 N) and, then, ddH₂O. The retentate was freeze-dried to obtain a dark orange powder (yield: 80-92%). The structures of the compounds were verified by NMR and GPC.

The syntheses of the triglycine and the triglycine-(PEG)₂₃ derivatives were achieved using the same protocol.

### Example 16: Synthesis of a H₂N-Gₙ-Cellophil-(Fluorescein)₈ or a H₂N-Gₙ-PEGₓ-Cellophil-(Fluorescein)₈

To a solution of BOC-G₃-Cellophil-(Fluorescein)₈ (1.0 eq.) or BOC-G₃-PEG-Cellophil-(Fluorescein)₈ (1.0 eq.) in EtOH was added a solution of HCl in EtOH (≈1.25 N). The resulting reaction mixture was stirred for 2 h at room temperature and dialyzed (MWCO 3.5 kDa) against water (2×). The retentate was freeze-dried to obtain a dark orange (electrostatic) powder (99%). The structures of the compounds were verified by NMR and GPC.

### Example 17: Synthesis of Cathepsin B-Sensitive Doxorubicin-Containing Cellophil

### Step 1 Preparation of H-Lys(Alloc)-OH

A solution of L-lysine hydrochloride (1.0 eq.) and basic copper (II) carbonate (1.1 eq.) in H₂O (100 mL) was refluxed for 30 min. Solids formed during reflux were removed by hot filtration. The filtrate was cooled to 0° C. and adjusted to pH 9 by the addition of solid sodium bicarbonate (3.1 eq.). Allyl chloroformate (1.5 eq.) was added dropwise over a period of 1 h, while the solution stirred at 0° C. During this addition, the reaction mixture was maintained at pH 9 by the addition of solid sodium bicarbonate. The reaction mixture was allowed to warm to RT and stirred overnight. The blue solid product formed during the reaction was collected by filtration in quantitative yield.

The solid copper salt of Lys(Alloc) was suspended in H₂O (250 mL), and 2 eq. of thioacetamide (2.0 eq.) were added. The alkaline suspension was stirred at 50° C. for 3 h, during which time the solid slowly dissolved. Subsequently, the solution was acidified to pH 2 with 2 M HCl, and was boiled for 5 min. The precipitated CuS was removed by filtration. The filtrate was concentrated under vacuum to about 60 mL, at which point the hydrochloride salt of Lys(Alloc) precipitated as a white solid (79%), which was recovered by filtration.

### Step 2 Preparation of FMOC-Val-Lys(Alloc)-OH

A vigorously stirred solution of FMOC-Val-OSu (1.0 eq.) in Dioxan (30 mL) at RT was combined with a solution of Lys(Alloc)-OH (1.1 eq.) and NaHCO₃ (2.1 eq.) in water (30 mL). The temperature was maintained below 25° C. using a cold water bath for the first 30 min. The mixture was continued to be stirred at room temperature for 14 h. The mixture was diluted with water (50 mL) and then acidified to pH 3 with 15% citric acid. The resulting suspension was extracted with ethyl acetate (3×100 mL), and the combined organic layers were washed with water and brine, dried, and evaporated to give an off-white solid. The solid was dissolved in THF and then methyl tert-butyl ether (MTBE) was added to give the pure white solid (80%) after filtration.

### Step 3 Preparation of FMOC-Val-Lys(Alloc)-PABOH

A stirred solution of FMOC-Val-Lys(Alloc)-OH (1.0 eq.) and PABOH (1.1 eq.) in THF (15 mL) at RT was treated with EEDQ (1.1 eq.). After 16 h, the mixture was evaporated to dryness at 30° C. and the residue was recrystallized from MTBE to give the deep yellow product (84%).

### Step 4: Preparation of H-Val-Lys(Alloc)-PABOH

FMOC-Val-Lys(Alloc)-PABOH (1.0 eq) in CH₂Cl₂ (35 ml) at room temperature was treated with diethylamine (50 ml). The mixture was sonicated briefly and stirred at room temperature for 4 h. The solvents were evaporated and the residue was flushed with CH₂Cl₂ and chromatographed on silica to give the product as a colorless foam (69%).

### Step 5: Preparation of MC-Val-Lys(Alloc)-PABOH

H-Val-Lys(Alloc)-PABOH (1.1 eq.) and DIEA (1.1 eq.) in CH₂Cl₂ (5 ml) at room temperature were treated with MC-NHS (1.1 eq.) in CH₂Cl₂ (2 ml). The mixture was stirred at room temperature overnight. Ethyl acetate (60 ml) was added, and the mixture was washed with water and brine, dried and evaporated to give the desired product (96%)

### Step 6: Preparation of MC-Val-Lys(Alloc)-PABO-PNP

A stirred solution of MC-Val-Lys(Alloc)-PABOH (1.0 eq.) in THF (15 mL) at room temperature was treated with PNP chloroformate (1.2 eq.) and dry pyridine (1.5 eq.). The reaction was stirred overnight until HPLC analysis indicated that no educt was present in the mixture. The mixture was diluted with EtOAc (50 mL) and acidified with citric acid (50 mL, 10% in H₂O). The organic layer was washed with water (50 mL) and brine (25 mL), dried over sodium sulfate, and the solvent was evaporated to give a light yellow solid. The solid was purified by flash chromatography on silica gel (20:1 DCM/MeOH) to give the pure compound as a yellowish solid (58%).

### Step 7: Preparation of MC-Val-Lys(Alloc)-PABC-DOX

MC-Val-Lys(Alloc)-PABO-PNP (1.0 eq.) and DOX-HCl (1.1 eq.) in NMP (8 mL) at RT were treated with Et₃N (1.1 eq.). The mixture was afterwards incubated in the dark over 3 days. The mixture was then diluted with 10% 2-Propanol/ethyl acetate (100 mL) and washed with water (3×100 mL) and brine (50 mL), dried, and evaporated to give a deep orange oil. This was purified by flash chromatography on silica gel to give the pure product as a red solid (92%).

### Step 8: Preparation of MC-Val-Lys-PABC-DOX

MC-Val-Lys(Alloc)-PABC-DOX (1.0 eq.) in THF (7 mL) under argon at RT was treated with Pd(PPh₃)₄ (0.03 eq.), acetic acid (2.5 eq.) and tributyltin hydride (1.5 eq.). The mixture was stirred at RT in the dark for 1 h during which time an orange solid began to form. The mixture was diluted with ether (25 mL) followed by the addition of 1 M HCl in ether (2 mL). The resulting suspension was sonicated briefly and then filtered. The orange solid was washed repeatedly with ether and then dissolved in 5:1 DCM:MeOH. To this was added Celite (7 g), and then the solvents? were evaporated. The resulting solid was adsorbed on Celite® and dry-loaded on a Celite-Column (from a slurry in 100:1 DCM:MeOH). The column was eluted with a mixture of 100:1 DCM:MeOH and then with a mixture of 10:1 DCM:MeOH. The desired product was obtained as an orange solid (30%).

Linker derivatives MC-Ala-Lys-PABC-DOX, MC-Val-Cit-PABC-DOX having different amino acid sequences were synthesized following the same procedures mentioned above with the exception that the citrulline linker did not need an alloc protection group.

### Step 9: Coupling of Cathepsin B Sensitive Linkers to a Cellophil Copolymer

The above mention linker-doxorubicin conjugates were subsequently coupled to Cellophil copolymer (of example 13, step 7) using the following general procedure:

To a solution of a Cellophil copolymer (1.0 eq.) and Et₃N (12 eq.) in DMF was added dropwise a solution of the linker-doxorubicin conjugate (10 eq.) at room temperature under an inert atmosphere. The reaction mixture was stirred overnight at room temperature. The mixture was finally diluted with ddH₂O and the resulting suspension filtered. The filtrate was then dialyzed (MWCO 3.5 kDa) against ddH₂O (2×10 L), and the retentate was lyophilized to obtain a red free flowing powder (60-70% yield). The structure of the obtained compound was verified by NMR spectroscopy.

### Example 18: Cathepsin B-Mediated Release of Doxorubicin from Cellophil Copolymer

Human Liver cathepsin B (Merck, MW ca. 27500) (5 units) was dissolved in 400 μL acetate buffer (50 mM acetate+1 mM EDTA, pH 5.0). 10 μL of enzyme solution was incubated with 390 μL of activating solution (5 mM dithiothreitol, 100 mM sodium phosphate buffer, 5 mM EDTA, 100 mM NaCl, 0.01% Brij58, pH 6.0) for about 30 minutes at 37° C. In the meantime, 20 μL of polymeric linker-Dox conjugate of example 17 (1 μmol) was added to 1473 μL of the activating solution and was incubated at 37° C. 32 μL of the activated Enzyme (0.01 U) were added to the substrate solution, and the reaction was incubated at 37° C. The release of free DOX was monitored via HPLC and photometer measurements. MC-Val-Lys-PABC-PNP-DOX exhibited the shortest half-life, followed by MC-Ala-Lys-PABC-DOX and MC-Val-Cit-PABC-DOX.

### Example 19: Copolymerization of Doxorubicin-Containing Co-Principle Monomers (AK-DOX-V1 and AK-DOX-V2)

The synthesis of Cellophil copolymer containing doxorubicin is achieved by the general procedure for the copolymerization of co-principle monomers (Example 13, step 7) using BOC-G-RAFT intermediate, BOC-Gₙ-DMA-RAFT pre-polymer, RAFT-PEGₓ-BOC or the corresponding BOC-deprotected reagent (obtained according to the protocol described in example 13, step 6) (1.0 eq.) as a RAFT agent and a doxorubicin-containing co-principle monomer (8.0 eq.) (AK-DOX-V1 or AK-DOX-V2).

### Example 20: Copolymerization of AK-Phenol

The synthesis of Cellophil copolymer containing iodine reactive monomers was achieved via the general procedure for the copolymerization of co-principle monomers (Example 13, step 7) using BOC-G₃-RAFT intermediate, BOC-G₃-DMA-RAFT pre-polymer, RAFT-PEG-BOC or the corresponding BOC-deprotected reagent (obtained following protocol described in example 13, step 6) (1.0 eq.) as RAFT reagent and AK-Phenol (8.0 eq.) as co-principle monomer. Instead of ddH₂O 0.1 M NaHCO₃ was used as solvent. The structure of the desired product was verified by NMR spectroscopy.

### Example 21: Iodination of Iodine Reactive Polymeric Carriers

To a solution of the AK-Phenol copolymer of example 20 (70 mg, 7.0 μmol) in PBS buffer (pH=7.4) were successively added NaI (7.86 mg, 52 μmol) and Chloramine T (14.8 mg, 52 μmol). The reaction mixture was then stirred for 30 min at RT and thereafter diluted with an aqueous solution of Na₂S₂O₅ (0.3 M). The resulting solution was then dialyzed against 10 L ddH2O and the retentate was freeze-dried. The structure of the desired product was verified by NMR spectroscopy.

### Example 22: Coupling of H₂N-G₅-Cellophil-(Fluorescein)₈ to a Her2+ Model Antibody Using a Sortase-Mediated Reaction

H₂N-G₅-Cellophil-(Fluorescein)₈ (example 16) can be conjugated to fully human monoclonal antibody against the Her2 antigen using the following general procedure. Her2 monoclonal antibody [10 μM] genetically modified with a sortase motif (LPETG) and a hexa-histidine-tag (His₆) at the C-terminus of its heavy chains is incubated with H₂N-G₅-Cellophil-(Fluorescein)₈ [100 μM] in the presence of 0.62 μM sortase A in 50 mM Hepes, 150 mM NaCl, 5 mM CaCl₂, pH 7.5 for 3.5 h at 25° C. The reaction is stopped by passing it through a Protein A HiTrap column (GE Healthcare) equilibrated with 25 mM sodium phosphate (pH 7.5). The loaded column is washed with 5 column volumes (CVs) of buffer. Bound conjugate is eluted with 5 CVs of elution buffer (0.1 M succinic acid, pH 2.8) with 1 CV fractions collected into tubes containing 25% (v/v) 1 M Tris-base to neutralize the solution. Protein-containing fractions are pooled and subsequently formulated in 10 mM sodium succinate pH 5.0, 100 mg/mL trehalose, 0.1% (w/v) polysorbate 20 by buffer exchange using NAP-25 columns (GE Healthcare) according to the manufacturer's instructions. Success of coupling reaction is verified qualitatively by SDS PAGE on 4-20% gradient Tris-glycine gels and by western blot (WB) analysis using an anti-His₆ primary antibody and a horse-radish peroxidase conjugated secondary antibody. Detection of WB signal is performed using an enhanced chemiluminescence (ECL) Kit (Pierce™, ECL Western Blotting Substrate). Non-modified anti-Her2-LPETG-His₆ antibody serves as control. Disappearance of anti-His₆ antibody indicates sortase reaction has run to completion. For quantitative analysis size exclusion chromatography is performed. Drug to antibody ratio (DAR) is calculated by comparison of peak intensities of residual non-modified antibody (UV detection wavelength 280 nm).

### Example 23: Exploratory Toxicity of a H₂N-G₃-Cellophil Copolymer

The polymeric carriers of this disclosure are not biodegradable. It was therefore important to demonstrate that the carriers are harmless for healthy tissues. An exploratory toxicity study of a polymeric carrier of the present disclosure (without payload) was performed. Briefly, HepG2 cells were plated on 96-well black walled, clear bottomed polystyrene plates at 100 μL per well. Test compound was a H₂N-G₃-Cellophil copolymer (12 kDa) containing DMA and AK (90/10 mol %) that was prepared by the procedure described in example 13, step 7. The HepG2 cells were dosed with test compound at concentrations from 0.04 to 100 μM. At the end of 72-h incubation at 37° C., appropriate dies or antibodies were added to the cultures. The plates were then scanned using an automated fluorescent cellular imager (ArrayScan®, Thermo Scientific Cellomics).

The following eight cell health parameters (CHP) were evaluated:

Cell count: A decreasing number of cells per well indicates toxicity due to necrosis, apoptosis or a reduction in cellular proliferation. Nuclear size: An increase in nuclear area can indicate necrosis or G2 cell cycle arrest, and a decrease can indicate apoptosis. DNA structure: An increase in DNA structure can indicate chromosomal instability and DNA fragmentation. Mitochondrial mass: A decrease in mitochondrial mass indicates loss of total mitochondria and an increase implies mitochondrial swelling or an adaptive response to cellular energy demands. Mitochondrial membrane potential (Δψm): A decrease indicates a loss of mitochondrial integrity, typically resulting in apoptotic signaling; an increase in mitochondrial membrane potential indicates an adaptive response to cellular energy demands. Oxidative stress: An increase in reactive oxygen species (ROS) is an early cytotoxic response. Glutathione content: A decrease in glutathione (GSH) content can result from production of ROS or from direct binding to tested compound. An increase in GSH content represents an adaptive cellular response to oxidative stress. Cellular ATP: After cell lysis, ATP is released from the cell. Cells which are not metabolically active will not release any ATP. Therefore, a decrease in metabolically active cells will result in a decrease in the level of ATP detected.

TABLE 2
Influence of H₂N-G3-Cellorthil copolymer on CHPs

| | MEC | AC50 | MTD | MEC [μM] | AC₅₀ [μM] | MTD [μM] |
|---|---|---|---|---|---|---|
| CHP | [μM] | [μM] | [μM] | control 1/2 | control 1/2 | control 1/2 |
| Cell count | NR | NR | NR | 1.73/15.5 | 2.54/25.7 | 2/40 |
| Nuclear size | NR | NR | | 3.76/NR | 8.09/NR | |
| DNA structure | NR | NR | | 0.63 | 46.7 | |
| | | | | (NS)/NR | (NS)/NR | |
| Mitochondrial | NR | NR | | NR/NR | NR/NR | |
| mass | | | | | | |
| Mitochondrial | NR | NR | | NR/NR | NR/NR | |
| membrane | | | | | | |
| potential | | | | | | |
| Oxidative stress | NR | NR | | 0.539/NR | 22.4/NR | |
| Glutathione | NR | NR | | NR/1.7 | NR/10.4 | |
| content | | | | | | |
| Cellular ATP | NR | NR | | 3.13/14.4 | 4.15/16.2 | |

Control 1: carbonyl cyanide 3-chlorophenylhydrazone;
Control 2: L-buthionine-sulfoximine;
MEC: Minimal effective dose, i.e., the lowest dose at which an effect is detected:
AC₅₀: Concentration at which 50% of the maximum effect is observed;
MTD: Maximal tolerated dose, i.e., the concentration at which <20% cell loss was observed.
NR: No response;
NS: not statistical significant.

This study revealed that exposure of the HepG2 cells to the G₃-Cellophil copolymer at concentrations of up to 100 μM had no effect on the tested CHP. This demonstrates the high biocompatibility of the copolymers of the present disclosure.

### Example 24: Cancer Cell Specificity of a Cellophil-(Fluorescein)ₙ-ADC

The affinity of the HER2-antibody-functionalized G₅-Cellophil-(Fluorescein)₈ copolymer of example 22 for its target cell is examined in an experiment using the SKBR3 and MDA-MB-468 cancer cell lines. SKBR3 cells overexpress the human epidermal growth factor receptor 2 (HER2+), whereas MDA-MB-468 cells do not express the receptor (HER2−). Binding is assessed by FACS (=fluorescence-activated cell sorting) using the following brief protocol:

Cells are plated in 96 well plates with a density of 5,000 to 10,000 cells in 160 μL medium/well [DMEM supplemented with 4.5 g/L glucose, 1.5 mM L-glutamine and 10% fetal bovine serum (MG-30, CLS)]. After one day incubation at 37° C. in a humidified incubator in a 5% CO₂ atmosphere, cells are harvested, washed and cell suspensions adjusted to a concentration of 1.25×10⁶ cells/mL in ice cold PBS (pH 7.5) supplemented with 10% fetal calf serum (FCS), 1% sodium azide. Cell suspensions are transferred to polystyrene round bottom 12×75 mm² and then incubated with 5 μg/mL Cellophil-(Fluorescein)₁₆-ADC for 45 min in the dark at 4° C. Thereafter, cells are washed 3 times by centrifugation at 400×g for 5 min and re-suspended in 500 μL ice-cold PBS (pH 7.5, supplemented with 10% FCS, 1% sodium azide) before being analyzed on a flow cytometer.

FACS staining of SKBR3 cells exposed to Cellophil-(Fluorescein)₁₆-ADC or to Fluorescein-tagged Trastuzumab is compared to demonstrate that the target affinity of the antibody in the Cellophil-Fluorescein-ADC is preserved. MDA-MB-468 cells are used to analyze non-specific binding of the Cellophil-ADC.

### Example 25: Attachment of a G₅-Cellophil-(Fluorescein)₈ to a Model Protein

A model protein (red fluorescent mCherry) was genetically modified with a sortase recognition motive (LPETG) at its C-terminus and an additional cysteine near its N-terminus. A DNA fragment containing the modified coding sequence for mCherry was synthesized in vitro:

Coding sequence Cys-mCherry-LPETG-His6
(SEQ ID NO: 1):
5′-ATGTGTGGTGGTAGCGGTGGTTCAGGTGGTTCTGGCGGTAGTGGT
GGCAGCATGGTTAGCAAAGGTGAAGAGGATAATATGGCCATCATCAAA
GAATTCATGCGCTTCAAAGTTCACATGGAAGGTAGCGTTAATGGCCAC
GAATTTGAAATTGAAGGTGAAGGCGAAGGTCGTCCGTATGAAGGCACC
CAGACCGCAAAACTGAAAGTTACCAAAGGTGGTCCGCTGCCGTTTGCA
TGGGATATTCTGAGTCCGCAGTTTATGTATGGTAGCAAAGCCTATGTT
AAACATCCGGCAGATATTCCGGATTACCTGAAACTGAGCTTTCCGGAA
GGTTTTAAATGGGAACGTGTGATGAATTTTGAAGATGGTGGTGTTGTT
ACCGTTACACAGGATAGCAGCCTGCAGGATGGTGAATTTATCTATAAA
GTTAAACTGCGTGGCACGAATTTTCCGAGTGATGGTCCGGTTATGCAG
AAAAAAACCATGGGTTGGGAAGCAAGCAGCGAACGTATGTATCCGGAA
GATGGCGCACTGAAAGGTGAAATTAAACAGCGTCTGAAGCTGAAAGAT
GGCGGTCATTATGATGCAGAAGTTAAAACCACCTACAAAGCCAAAAAA
CCGGTTCAGCTGCCTGGTGCATATAACGTTAACATCAAACTGGATATT
ACCAGCCACAACGAGGATTATACCATTGTGGAACAGTATGAACGTGCA
GAAGGTCGCCATAGTACCGGTGGTATGGATGAACTGTATAAAGGTGGC
AGTGGTGGATCTGGTGGCTCAGGCGGAAGCGGTGGTAGCCTGCCGGAA
ACCGGTGGTCTGAATGATATTTTTGAAGCCCAGAAAATCGAATGGCAT
GAACATCATCACCATCACCACTAA-3′

This DNA fragment was subcloned into cloning vector pMA-T (Invitrogen/Thermo Fisher Scientific, Germany). The latter construct was digested with NdeI and BamH1. The digest was electrophoresed on a 1.5% agarose gel, and the mCherry DNA-containing fragment was excised and the DNA extracted using a Qiagen gel extraction kit (Qiagen, Hilden, Germany). The purified DNA fragment was then ligated into the expression vector pET28-c using T4 DNA ligase (New England Biolabs, UK). The correctness of inserted DNA sequence was subsequently verified by sequence analysis. For protein production, the resulting plasmid (pCIS-[C]-mcherry-[LPETG]) was transformed into competent Escherichia coli BL21DE3 cells. Cells were grown (LB medium+Ampicillin 100 μg/ml, 37° C., 500 ml shaking flasks) to an OD₆₀₀ of 0.4 before protein expression was induced with 1 mM Isopropyl-3-D-thiogalactopyranoside (IPTG). Cells were harvested 4 h later by centrifugation (6000×g, 15 min, 4° C.), suspended in lysis buffer (50 mM NaH₂PO₄, 0.5 M NaCl, pH 8.0) and lysed by sonication. Cells debris were removed by centrifugation (30,000×g, 30 min, 4° C.). Purification of Cys-mcherry-LPETG-His₆ protein was performed by Nickel-NTA affinity chromatography (Nickel-NTA Agarose, Thermo Fisher Scientific, Germany) according to the manufacturer's protocol. Protein yield was quantified by Bradford assay (Bio-Rad Laboratories GmbH, München, Germany), and size and purify of the recombinant protein were verified by SDS-PAGE.

Purified LPETG-tagged mCherry [10 μM] is subsequently incubated with different concentrations of H₂N-G₅-Cellophil-(Fluorescein)₈ of example 16 [20-100 μM] in the presence of increasing concentrations of Sortase A [0.062-0.62 μM] in 50 mM Hepes, 150 mM NaCl, 5 mM CaCl₂, pH 7.5 for 3.5 h at 25° C. Control reactions that lack Cellophil copolymer or Sortase A are run in parallel. Reactions (20 μl) are stopped by the addition of 5 μl 4×SDS-PAGE loading buffer+10% w/v β-mercaptoethanol (Biorad, Germany) and heat treatment (5 min, 95° C., constant shaking at 600 rpm). The samples are then electrophoresed on 4-20% SDS-PAGE gels (Mini-PROTEAN® TGX™ Precast Gels Biorad, Germany) at 150 V for 30 min, and the gels are subjected subsequently to Coomassie blue staining. Success of coupling is estimated based on the appearance of a product with a larger size than mCherry.

### Example 26: Functionalization of Cellophil-(Fluorescein)₈ with Tumor Cell-Specific Aptamer DML-7

### Step 1: Synthesis of 1-((3-azidopropyl)amino)-2-methyl-1-oxopropan-2-yl ethyl carbonotrithioate

To a solution of 2,5-dioxopyrrolidin-1-yl 2-(((ethylthio)carbonothioyl)thio)-2-methylpropanoate (2.4 g, 7.47 mmol, 1.0 eq.) and Et₃N (1.249 mL, 8.96 mmol, 1.2 eq.) in CH₂Cl₂ (44 mL) a solution of 3-azidopropan-1-amine (0.879 mL, 8.96 mmol, 1.2 eq.) in CH₂Cl₂ (15 mL) was added dropwise during 60 min at room temperature and under an inert atmosphere. The reaction mixture was stirred overnight at room temperature. Finally, the reaction mixture was successively washed with an aqueous solution of HCl (1 M, 3×20 mL), ddH₂O (2×25 mL) and an aqueous saturated solution of NaHCO₃ (20 mL). The organic phase was dried (Na₂SO₄), and the volatiles were removed under reduced pressure. The residual orange oil (2.25 g, 7.34 mmol, 98%) was used without further purification. The structure of the title compound was verified by NMR spectroscopy.

### Step 2: Synthesis of RAFT-DMA-N₃ Pre-Polymer

The synthesis of the title compound was achieved following the general protocol for pre-polymer synthesis of example 13, step 5, and using 1-((3-azidopropyl)amino)-2-methyl-1-oxopropan-2-yl ethyl carbonotrithioate as a starting material. The structure of the titled compound was verified by MS and NMR spectroscopy.

### Step 3: Synthesis of Cellophil-N₃

The synthesis of the title compound was achieved following the general protocol of example 13, step 7, using RAFT-DMA-N₃ pre-polymer as starting material. The structure of the title compound was verified by NMR spectroscopy.

### Step 4: Synthesis of Cellophil-(Fluorescein)₈-N₃

The synthesis of the titled compound was achieved following the general protocol for amine-reactive active agents of example 15, using Cellophil-N₃ and Fluorescein-NHS as starting materials. The structure of the title compound was verified by NMR spectroscopy.

### Alternative Synthesis

The synthesis of Cellophil copolymer containing fluorescein can be achieved via the general procedure for the copolymerization of co-principle monomers (example 13, step 7), using RAFT-DMA-N₃ pre-polymer as RAFT reagent and AK-Fluorescein-V2 as co-principle monomer (8.0 eq.)

### Step 5: Synthesis of Aptamer DML-7-[C6]-NH₂

A modified form of aptamer DML-7 (Duan et al. (2016) Oncotarget 7(24): 36436), known to be specific for metastatic prostate cancer cells, was synthesized on solid phase (Sigma Aldrich, Gillingham, UK):

(SEQ ID NO: 2)
5′-ACGCTCGGATGCCACTACAGGTTGGGGTCGGGCATGCGTCCGGA
GAAGGGCAAACGAGAGGTCACCAGCACGTCCATGAG-3′-[C6]-NH₂

A six-carbon atom spacer and a reactive amino group had been added to facilitate functionalization. The lyophilized powder of the aptamer was afterwards rehydrated in (buffer) for 2 h at room temperature. Subsequently, the solution was heated to 95° C. during a 10-min period and then allowed to cool down to room temperature over-night to obtain the correct three-dimensional conformation.

### Step 6: Coupling of DML-7-[C6]-NH₂ Aptamer to Cellophil-(Fluorescein)₈-N₃

To a solution of DML-7-[C6]-NH₂ (1.0 eq., prepared in step 5) in DNAase-free PBS buffer is added a Dibenzocyclooctyne-N-hydroxysuccinimidyl ester (1.5 eq.). The reaction mixture is mixed at room temperature until full conversion of aptamer amine is observed (LC-MS). Subsequently, Cellophil-(Fluorescein)₈-N₃ (2.0 eq.) is added and allowed to react until full conversion of alkyne-aptamer intermediate is observed. The mixture is then purified by semi-preparative GPC with water (+0.01% NaN₃) as eluent. The purified fraction containing the desired product is desalted using a desalting column (PD10, Thermo Fisher Scientific, German). Subsequently the desired product is lyophilized to obtain a white powder.

The resulting aptamer-containing Cellophil-(Fluorescein)₈ is analyzed by electromobility shift assay (EMSA). For this analysis, to 18 μL of a stock solution of the latter copolymer [0.3 mg/mL in EMSA buffer (10 mM Tris-HCl, 75 mM KCl, 0.25 mM EDTA, 0.1% Triton X100, 5% glycerol (v/v), 0.2 mM DTT, pH 8.0)] 2 μL 5× nucleic acid sample buffer (Biorad, Germany) are added. Subsequently, the sample is electrophoresed on a 1.5% agarose gel (supplemented with 0.25 μg/ml ethidium bromide for UV staining, 1×TAE buffer, 135 V, 35 min). DML-7 aptamer and Cellophil-(Fluorescein)₈-N₃ serve as controls. A shift in band migration clearly identifies the increase in the molecular weight of the aptamer due to the covalent attachment of the Cellophil-(Fluorescein)₈ moiety.

### Example 27: Synthesis of an Oxime Functionalized Polymeric Carrier

### Step 1: Synthesis of tert-butyl (6,6-dimethyl-7,12-dioxo-4-thioxo-3,5-dithia-8,11-diazatridecan-13-yl)oxycarbamate (RAFT-EDA-Oxime-BOC)

To a solution of 2-(((tert-butoxycarbonyl)amino)oxy)acetic acid (486 mg, 2.54 mmol, 1.0 eq.) in CH₂Cl₂ (40 mL) was added HOBt hydrate (467 mg, 3.05 mmol) and N₁-((ethylimino)methylene)-N3,N3-dimethylpropane-1,3-diamine hydrochloride (511 mg, 2.67 mmol, 1.05 eq.) at 0° C. under N₂. The resulting solution was stirred for 30 min at 0° C. Finally 2-(2-(((ethylthio)carbonothioyl)thio)-2-methylpropanamido)ethanaminium 2,2,2-trifluoroacetate (966 mg, 2.54 mmol, 1.0 eq.) and N-ethyl-N-isopropylpropan-2-amine (2.24 mL, 13.2 mmol, 5.2 eq.) were successively added, and the reactive mixture was stirred for 1 h at 0° C. and then overnight at room temperature. All volatiles were removed under reduced pressure, and the residue was taken up in EtOAc (100 mL). The organic mixture was successively washed with an aqueous saturated solution of NH₄Cl (3×40 mL), an aqueous saturated solution of NaHCO₃ (3×40 mL), ddH₂O (3×40 mL) and brine (40 mL). The organic phase was dried (Na₂SO₄) and all volatiles removed under reduced pressure. The resulting yellow solid residue was suspended in Et₂O (40 mL). The suspension was filtered and the filter cake was washed with Et₂O (2×20 mL), ddH₂O (3×20 mL) and Et₂O (3×20 mL) and dried under vacuum. The product was isolated (900 mg, 2.00 mmol, 79% yield) as a yellow powder. The structure of the title compound was verified by NMR spectroscopy.

### Step 2: Synthesis of RAFT-DMA-Oxime-BOC Pre-Polymer

The synthesis of the title compound was achieved following the general protocol for pre-polymer synthesis of example 13, step 5, and using RAFT-EDA-Oxime-BOC as a starting material. The structure of the desired product was verified by MS and NMR spectroscopy.

### Step 3: Synthesis of Cellophil-Oxime-BOC

The synthesis of the titled compound was achieved following the general protocol of example 13, step 7, using RAFT-DMA-Oxime-BOC pre-polymer as a starting material. The structure of the title compound was verified by GPC and NMR spectroscopy.

### Step 4: Synthesis of Cellophil-(Fluorescein)₈-Oxime-BOC

The synthesis of the title compound can be achieved following the general protocol for amine-reactive active agents of example 15 and using Cellophil-Oxime-BOC and FITC as starting materials. The structure of the title compound can be verified by NMR spectroscopy.

### Step 5: Synthesis of Cellophil-(Fluorescein)₈-Oxime

Cellophil-(Fluorescein)₈-Oxime-BOC is deprotected following the general procedure for BOC deprotection (Example 16).

### Step 6: Coupling of Cellophil-(Fluorescein)-Oxime to Aldehyde-IgG

Cellophil-(Fluorescein)₈-Oxime can be covalently coupled to oxidized (NaIO₄) polyclonal antibody IgG (aldehyde-IgG) as described in the literature (Dong et al. (2017) Angew Chem Int Ed 56: 1273). The structure of the desired product can be verified by MS.

### Example 28: Attachment of Cellophil-(Fluorescein)-N₃ to a Model Protein

### Step 1: Alkyne Functionalization of the mCherry Model Protein

To a solution of cysteine-bearing mCherry model protein of example 25 (1.0 eq.) in degassed PBS buffer, pH 7.5, is added an excess of tris(2-carboxyethyl)phosphine (TCEP) (100 eq.) under an inert atmosphere. The resulting solution is thoroughly mixed and allowed to rest for 20 min before a degassed solution of dibenzocyclooctyne-maleimide (DBCO-maleimide) in DMSO is added under inert atmosphere. The resulting reaction mixture was stirred overnight at room temperature. The mixture is then purified by semi-preparative GPC with water (+0.01% NaN₃) as eluent. The purified fraction containing the desired product is desalted using a desalting column to obtain mCherry-DBCO.

### Step 2: Cellophil Functionalization of the mCherry Model Protein Via Click Chemistry

A solution of mCherry-DBCO (1.0 eq.) and Cellophil-(Fluorescein)₈-N₃ (2.0 eq. obtained from example 26, step 4) in PBS buffer, pH 7.5, is stirred at room temperature for 16 h. The mixture is then purified by Ni²⁺ NTA affinity chromatography (Nickel-NTA Agarose, Thermo Fisher Scientific, Germany) according to the manufacturer's protocol. (mCherry contains a hexa-histidine tag.) The purified fraction containing the desired product is desalted using a desalting column to obtain mCherry-Cellophil(Fluorescein)₈. The resulting protein-polymer conjugate can be analyzed by SDS-PAGE using the protocol presented in example 25 and Cys-mcherry-LPETG-His₆ (see example 25) as control. An increased size of the product compared to the control observed in the Coomassie-stained gel indicates successful coupling of the polymer to the protein.

### Example 29: Functionalization of a Native Lysine Residue in a Her2+ Antibody (Trastuzumab) with the Amine-Modified NH₂-Gₙ-Cellophil-(DOX)₈ Utilizing an Aza-Michael Ligation Strategy

The synthesis of Trastuzumab-Cellophil-(DOX)₁₆ is achieved following the protocol developed by Bernades, G. J. L. (J Am Chem Soc (2018) 140: 4004-) using NH₂-Gₙ-Cellophil-(DOX)₈ as amine nucleophile that is coupled to the light chains of the antibody. This results in an ADC complex containing, on average, 16 doxorubicin molecules per antibody molecule. Trastuzumab (20 mg/ml in PBS) was obtained from Carbosynth, UK.

### Example 30: Anti-Cancer Efficacy of Trastuzumab-Cellophil-(DOX)₁₆

The anti-cancer efficacy of Trastuzumab-Cellophil-(DOX)₁₆ (see example 29) can be verified as follows:

The experiment is performed in 96-well plates using cancer cell lines SKBR3 and MDA-MB-468. SKBR3 cells overexpress the human epidermal growth factor receptor 2 (HER2+), whereas MDA-MB-468 cells do not express this receptor (HER2−). Wells are seeded with 5,000 to 10,000 cells in 75 μL Dulbecco's Modified Eagle's medium (DMEM) supplemented with 4.5 g/L glucose, 1.5 mM L-glutamine and 10% fetal bovine serum (MG-30, CLS). After one day of incubation at 37° C. and 5% CO₂ in a humidified incubator, serial dilutions of ADC Trastuzumab-Cellophil-(DOX)₁₆ (prepared in example 29) in 25 μl growth medium are added to the wells. Final ADC concentrations in the wells range from 0.02 ng/mL to 20 μg/mL (ADC-naive cells serving as negative controls). Each dilution is tested in triplicate. For comparison purposes, parallel cultures receive serial dilutions of Trastuzumab in growth medium. After 72 h of incubation, plates are removed from the incubator and equilibrated to room temperature. After approximately 20 minutes, cell viability is assessed by aWST-1 cell proliferation assay (Sigma-Aldrich, Germany) that is performed according to the manufacturer's instructions. The readout of the assay is absorbance at 420-480 nm. The anti-cancer efficacy of Trastuzumab-Cellophil-(DOX)₁₆ is estimated by comparing absorbance values measured in ADC-treated, not-treated and Trastuzumab-treated cultures, respectively. Comparison of results obtained with SKBR3 and MDA-MB-468 will inform about the target specificity of the ADC.

### Example 31: Synthesis of a Click-Reactive Azide-Cellophil(Fluorescein)₈ Using Fluorescein-Modified Co-Principle Monomers

AK-Fluorescein (Example 10) was copolymerized in a RAFT polymerization with an azide-modified RAFT agent (1-((3-azidopropyl)amino)-2-methyl-1-oxopropan-2-yl ethyl carbonotrithioate) utilizing the following protocol:

DMA (0.97 mmol, 80 eq.) and AK-Fluorescein (0.097 mmol, 8 eq.) were dissolved in 2 ml dry dioxane, and N₃—RAFT [(1-((3-azidopropyl)amino)-2-methyl-1-oxopropan-2-yl ethyl carbonotrithioate] (0.012 mmol, 1 eq.) and AIBN (4.85 μmol, 0.4 eq.) were added. After complete dissolution, polymerization was induced by heating to 65° C. Polymerization was completed after 6 h of incubation at 65° C. Subsequently, the reaction mixture was cooled to room temperature, and the RAFT group of the copolymer was removed using the protocol presented in example 13. The resulting mixture was then dialyzed (MWCO 3.5 kDa) against ddH2O, and the retentate was lyophilized to obtain N₃-Cellophil(Fluorescein)₈ as an orange powder (85%). The structure of the compound was verified by NMR spectroscopy and GPC analysis (MW of about 13 kDa, PDI of 1.08)

N₃-Cellophil(Fluorescein)₈ can be used in a copper-free click reaction with an alkyne (e.g. DBCO)-modified cancer cell-specific targeting moiety.

### Example 32: Synthesis of Iodine-Loaded Cellophil-Antibody Conjugate Targeting an Oncogenic Protein

In order to generate a model antibody polymer conjugate against a widespread oncogene, a commercially available antibody targeting the protein BMI-1 was chosen. BMI-1 (polycomb ring finger oncogene) is necessary for efficient self-renewing cell divisions of adult hematopoietic stem cells as well as adult peripheral and central nervous system neural stem cells. BMI-1 has been reported as an oncogene regulating p16 and p19, which are cell cycle inhibitor genes. Overexpression of BMI-1 seems to play an important role in several types of cancer, such as bladder, skin, prostate, breast, ovarian, colorectal as well as hematological malignancies (Lessard J et. al. (2003). Nature 423 (6937): 255-60. doi:10.1038; Molofsky A V et. al. (2005). Genes Dev. 19 (12): 1432-7. doi:10.1101/gad.1299505)

Synthesis of NH₂-GGG-Cellophil [DMA₄₁/AK-Phenol₃] which is capable of being loaded with radioactive iodine was performed in analogy to protocols presented in example 20 but with adjusted molar ratios between AK-Phenol (3 eq.) and principle monomers DMA (41 eq). The mean molecular weight (5.6 kDA) and molecular weight distribution (PDI 1.18) of the resulting copolymer was documented by LC-MS and gel permeation chromatography. After purification by dialysis and freeze drying, the Cellophil copolymer was coupled to an AbFlex™ BMI-1 (monoclonal) antibody against the full length human polycomb ring finger oncogene containing a sortase recognition motif (LPETG) using the following protocol:

BMI-1 antibody [6 μM] was incubated with NH₂-GGG-Cellophil [DMA₄₁/AK-Phenol₃][120 μM] in the presence of [2 μM] sortase A (Sortase A5 protein [S. aureaus, Uniprot A0A077UNB8-1] containing amino acid substitutions P94R, D160N, D165A, K190E and K196T, and including a C-terminal 6×His-Tag; Active Motif Inc., USA). in a HEPES-based reaction buffer (Active Motif Inc., USA) for 1 h at 30° C. U.S. Pat. No. 9,267,127. Control reactions that lack Cellophil copolymer or Sortase A were run in parallel. The calcium-dependent coupling reaction was stopped by the addition of EDTA disodium salt (250 mM) to a final concentration of 5 mM, and samples were stored at 4° C. until final characterization.

For analysis, the antibody-polymer conjugate was digested with Fabricator® (Genovis Inc., USA). [FabRICATOR (IdeS) is a cysteine protease that digests antibodies at a specific site below the hinge, generating a homogenous pool of F(ab′)₂ and Fc/2 fragments]. Digestion was performed according to manufacturer's protocols. Subsequently, the cleavage products were analyzed by SDS-PAGE using the protocol of example 25. Shift to higher molecular weight in the Fc/2 band of the antibody indicated successful coupling of the Cellophil copolymer to the antibody. The efficiency of the coupling reaction was analyzed in a semi-quantitative manner by comparison with the residual (non-modified) Fc/2 band of the negative control. The coupling efficiency was found to be about 50%.

For detailed characterization of coupling products, an LC-MS analysis of the IdeS-digested antibody-Cellophil conjugate was performed using the following protocol:

The reaction mixture of the IdeS-digested antibody-Cellophil conjugate was diluted 10 times with ddH₂O. 5 μL of the resulting solution were injected into the LCMS system (G6230 LC-MS TOF System, Agilent, Santa Clara, Calif.) and separated using an C8-HPLC column with an eluent consisting of water, isopropanol, ACN and 0.1% FA. Subsequently, chromatograms and spectra were deconvoluted using Agilent's Masshunter software solution. Analysis of chromatograms and spectra indicated that the copolymer was coupled only to the heavy chain of the mAB.

The Cellophil-anti-BMI-1 conjugate can be loaded with iodine radioisotopes using a protocol presented in example 21 to generate an antibody-polymer-conjugate for targeted cancer therapy. In some embodiments, e.g. in the case of an iodine isotope with a long half-life, the loading of the AK-Phenol-containing copolymer may be performed prior to coupling to the targeting antibody.

### Example 33: Synthesis of a Cellophil (DMAₙ/AKₘ) Copolymer with an mTG Tag (═NH₂-PEGₙ)-RAFT-BOC

The following procedure describes the synthesis of a Cellophil copolymer capable of being functionalized with a covalently attached chelating agent for binding radioisotopes. The here presented copolymer (mTG-tag)-DMA₃₀/AK₈ serves to illustrate the general synthesis procedure. Copolymer size and the number of sites for functionalization contained in the copolymer may be altered by changing the molar ratios of the monomers employed.

To a solution of DMA (116 μL, 1120 μmol, 30 eq.) and AK (60 mg, 300 μmol, 8 eq.) in ddH₂O were successively added tert-butyl (6,6-dimethyl-7-oxo-4-thioxo-11,14,17,20,23-pentaoxa-3,5-dithia-8-azapentacosan-25-yl)carbamate (22 mg, 32.2 μmol, 1.0 eq.) and VA044 (3.6 mg, 11.2 μmol, 0.3 eq.). The reaction mixture was stirred for 4 h at 60° C. The reaction mixture was then diluted with ddH₂O and dioxane. To this solution were successively added phosphinic acid (50 w %, 27 μL, 158 μmol, 5 eq.), TEA (22 μL, 158 μmol, 5 eq.) and AIBN (1.6 mg, 9.5 μmol, 0.3 eq.). The reaction mixture was stirred for 8 h at 75° C. The resulting mixture was then dialyzed (MWCO 3.5 kDa) against ddH₂O and the retentate was freeze-dried to obtain Cellophil BOC—NH-PEG₅-(DMA₃₀/AK₈) as a white powder (140 mg, 120 μmol, 78% over two steps). The structure of the obtained compound was verified by NMR spectroscopy and GPC using the following protocol: a stock solution of 3.33 mg/mL copolymer was prepared in elution buffer (deionized water containing 0.05% (w/v) NaN₃) and filtered through a 0.45 μm syringe filter. Subsequently, 0.4 mL of stock solution was injected in the port of the GPC device (1260 Infinity LC-System, Agilent, Santa Clara, Calif.). Chromatography was performed at a constant flow rate of 0.5 mL/min in elution buffer. Copolymer samples were separated on a Suprema three-column system (pre-column, 1000 Å, 30 Å; 5 μm particle size; PSS, Mainz, Germany) which was placed in an external column oven at 55° C. Copolymers were analyzed by RI (refractive index) and UV detectors. A calibration curve (10 points) was established using a pullulan standard. Molecular weights of characterized copolymers were estimated with reference to this standard.

### Example 34: Functionalization of Cellophil [BOC—NH-PEG₅-(DMA₃₀/AK₈)] Copolymer with Anhydride-DOTA/NHS-DOTA

To a solution of Cellophil BOC—NH-PEG₅-(DMA₃₀/AK₈) (20 mg, 3.45 μmol) in ddH₂O a solution of Anhydride-DOTA (25 mg, 36 μmol) or NHS-DOTA (27 mg, 36 μmol) in DMSO was added and stirred for 24 h at 35° C., to this solution was then added 3M HCl and heated to 0° C. for 1 h. The resulting mixture was then dialyzed (MWCO 3.5 kDa) against ddH₂O and the retentate was freeze-dried to obtain NH₂—PEG₅-(DMA₃₀/AK-DOTA₈). The structure of the obtained compound was verified by NMR spectroscopy.

### Examples 35: Synthesis of Cellophil [DBCO—NH-PEG₅-(DMA₃₀/AK-DOTA₈)]

A solution of Cellophil NH₂—PEG5-(DMA₃₀/AK-DOTA₈) (3.6 μmol) and DBCO-NHS (18 μmol) and Triethylamine (7.2 μmol) in dry DMSO (1.5 mL) was stirred for 24 h at 25° C. The resulting mixture was then dialyzed (MWCO 3.5 kDa) against 0.1M ammoniumcarbonate, and the retentate was freeze-dried to obtain Cellophil DBCO—NH-PEG5-(DMA₃₀/AK-DOTA₈). The reaction was followed via GPC, and the structure of the obtained compound was verified by NMR spectroscopy.

### Example 36: Synthesis of a Radiolabeled Trastuzumab-[Cellophil-(DOTA₄]₂ Conjugate for Diagnostic and Therapeutic Targeting Her2 Receptor Overexpressing Cancer Cells

In tumor diagnosis, the detection limit for the primary tumor or its metastasis is crucial for the survival rate of patients since late stage tumors are often associated with a poor prognosis. Using radiolabeled tumor tissue-specific antibodies for detection as well as subsequent therapy of cancer cells is a potentially promising method for radio medicine. However, approaches of this type have been hampered by low signal to noise ratios due to the facts that only a few radioisotopes could be attached to a targeting moiety/antibody and that the radioisotopes of interest have short half-lives (usually shorter than the half-life of the antibody). Therefore, an increased cargo of radioisotopes would be highly desirable. In this example, a radiolabeled antibody-Cellophil conjugate for improved tumor cell detection and therapy is described.

A DBCO-functionalized Cellophil polymer synthesized by procedures presented in example 33-35 is conjugated to a cancer cell-specific antibody of the IgG type (e.g., Trastuzumab for targeting Her2+ cancer cells) that has been functionalized with an azide-group at the glutamine in position 295 (Q 295) by a procedure described by Dennler et al. (Bioconjugate Chem. (2014) 25: 569-578).

Briefly, the antibody is deglycosylated by PNGaseF (Merck KGaA, Darmstadt, Germany). A reaction mixture containing 1 unit of enzyme per 10 μg Trastuzumab (Carbosynth Ltd, Berkshir, UK) in PBS (pH 7.4) is incubated overnight at 37° C. in order to activate Q295. Subsequently, deglycosylated Trastuzumab (6.6 μm) in PBS (pH 8) is incubated with NH₂-PEG₅-Azide (80 molar eq.) and microbial transglutaminase (MTGase) (6 U/mL, Zedira, Darmstadt, Germany) for 16 h at 37° C. After incubation MTGase activity is blocked by the addition of MTGase reactionstopper (Zedira, Darmstadt, Germany). To remove excess NH₂-PEG₄-Azide, MTGase and residual PNGaseF, the reaction mixture is buffer-exchanged (three times) into NH₄OAc (0.5 m, pH 5.5) by using an Amicon® Ultra 4 mL column (100 kDa MWCO, Merck KGaA, Darmstadt, Germany).

The actual click reaction is subsequently performed by incubation of Trastuzumab-(NH-PEG₅-Azide)₂ with a 3-fold molar excess of DBCO-functionalized Cellophil polymer for 3 h at 37° C., yielding Trastuzumab-(Cellophil-DOTA₄)₂. Excess polymer and non-functionalized Trastuzumab can be removed by size exclusion chromatography (SEC) and pooling of the fractions containing fully functionalized antibody.

Radiolabeling of the antibody-Cellophil conjugate with 111-InCl₃ (4 MBq per μg Trastuzumab-(Cellophil-DOTA₄₂ is performed for 1 h at 37° C., after which the indium-111-labeled antibody-polymer-conjugate is purified by SEC on a Superdex 75 10/300 GL column (GE Healthcare, Chicago, USA) run at a 0.5 mL/min flow rate. Major peak fractions are pooled. The resulting Trastuzumab-[Cellophil-(DOTA-In-111)₄]₂ may be used to detect Her2+ cancer cells by positron-emission-tomography (PET), e.g., in breast, colon or lung cancer patients, with a higher sensitivity than could be attained by conventional antibody-radioisotope complexes. The increased sensitivity is due to the increase In-111 cargo compared to a conventional radiolabeled antibody.

The same procedure may be used to prepare a therapeutic antibody-Cellophil-conjugate loaded with a suitable therapeutic radioisotope such as Lutecium-177 [substituting the 111-InCl₃ used in the above-described procedure with 177-LuCl₃].

### Example 37: Synthesis of a Cycloalkyne-Cellophil(DOTA_{J})-CO₂H

To functionalize a copolymer of this disclosure with a click-reactive cycloalkyne group, e.g. DBCO, the click-reactive moiety may be integrated during the removal of the RAFT group.

### Step 1—Synthesis of the Cycloalkyne-Initiator

A DBCO-modified Initiator is synthesized following the protocol of Ulbrich and co-workers (Polym. Chem., 2014 5, 1340).

### Step 2—Synthesis of RAFT-Cellophil-CO₂H Copolymer

Synthesis of a RAFT-Cellophil-CO₂H copolymer is achieved following the general protocol described in Example 13 (starting from the Ethyl-RAFT reagent Step 6-7).

### Step 3—Synthesis of cycloalkyne-Cellophil(DOTA)-CO₂H Copolymer

To a solution of RAFT-Cellophil-CO₂H in DMSO/ddH₂O (1/1) is added cycloalkyne-containing initiator (20 eq.) in one portion. The reaction mixture is sealed and heated at 70° C. until the disappearance of the yellow color (4 hrs). The reaction progress is followed by HPLC as well. The resulting solution is cooled to RT, and the pH is adjusted to 8 before p-NCS-Bz-DOTA-GA (Chematech) or DOTA-NHS is added (2 eq. per reactive amino group in the copolymer). The mixture is dialyzed against ddH₂O (MWCO: 5000 Da), and the retentate is lyophilized and characterized by NMR spectroscopy and SEC.

### Example 38—Synthesis of a Radiolabeled Trastuzumab-[Cellophil-(DOTA₄]₄ Conjugate for Therapeutic Targeting of Her2 Receptor-Overexpressing Cancer Cells

DBCO-functionalized Cellophil polymer synthesized by procedures presented in example 33 & 35 was conjugated to a cancer cell-specific antibody of the IgG type (Trastuzumab® for targeting Her2+ cancer cells) that had been functionalized with 2 azide groups per heavy chain (resulting in the addition of up to 4 azide groups per Antibody) means of a commercially available enzyme-based modification kit (SiteClick™ Antibody Labeling System, Thermo-Fisher-Scientific, Waltham, USA) used according to the manufacturer's protocol.

Coupling to the azide groups in Trastuzumab-azide₄ was realized according to the protocol presented in example 36 using a 1.5-fold molar excess of DBCO-Cellophil copolymer to azide groups in the antibody. Successful coupling was verified by SDS-PAGE.

Radiolabeling with 177-LuCl₃ [8 MBq per μg Trastuzumab-(Cellophil-DOTA₄)₄] is performed by incubation for 1 h at 37° C., after which the Lutecium-177-labeled antibody-polymer-conjugate is purified by SEC on a Superdex 75 10/300 GL column (GE Healthcare, Chicago, USA). The resulting Trastuzumab-[Cellophil-(DOTA-Lu-177)₄]₄ can be used to target and destroy Her2-overexpressing cancer cells.

The same procedure may be used to prepare a diagnostic antibody-Cellophil conjugate, substituting radioisotope Lu-177 with a suitable diagnostic radioisotope, e.g., Gallium-68 [by substituting 68-GaCl₃ for 177-LuCl₃ in the above-presented procedure].

### Example 39: Synthesis of a Radiolabeled Trastuzumab-[Cellophil-(DOTA₈]₂ Conjugate for Diagnostic and Therapeutic Targeting of Her2 Receptor-Overexpressing Cancer Cells from mTg-Tag Cellophil

The Cellophil copolymer of example 34 may be directly coupled to a monoclonal antibody like Trastuzumab by a transglutaminase-mediated reaction by using the NH₂—PEG₅ group in the copolymer as the substrate. For this, the protocol of example 36 is used with the exception that the NH2-PEG₄-Azide is replaced by NH₂—PEG₅-DMA₃₀/AK-DOTA₈ used at a 40-fold molar excess over antibody to generate an antibody with 16 chelating agents, i.e., Trastuzumab-[Cellophil-(DOTA₈]₂.

### Example 40: Synthesis of a Tetrazine-Functionalized Cellophil Copolymer for Coupling to a Targeting Moiety by TCO-Tz Click Chemistry

### Step 1: Synthesis of Tetrazine-Cellophil Copolymer

A solution of Cellophil copolymer of example 34 (3.6 μmol) and 2,5-dioxopyrrolidin-1-yl 2-(4-(6-methyl-1,2,4,5-tetrazin-3-yl)phenyl)acetate (18 μmol) and Triethylamine (7.2 μmol) in dry DMSO (1.5 mL) was stirred for 24 h at 25° C. The resulting mixture was then dialyzed (MWCO 3.5 kDa) against 0.1M ammoniumcarbonate, and the retentate was freeze-dried to obtain tetrazine-Cellophil [DMA₃₀/AK-DOTA₈). The reaction was followed by SEC, and the structure of the obtained compound was verified by NMR spectroscopy.

### Example 41: Synthesis of a Fluorophore-Modified Cellophil-[DMA₃₀/AK-DOTA) by Utilizing a Tetrazine-Strained Alkyne [4+2] Cycloaddition)

A solution of tetrazine-modified Cellophil copolymer of example 40 (1.3 μmol) and (E)-6-amino-9-(2-carboxy-5-((5-(((cyclooct-4-en-1-yloxy)carbonyl)amino)pentyl)carbamoyl)phenyl)-4,5-disulfo-3H-xanthen-3-iminium (1.3 μmol) in dry DMSO (0.5 mL) was stirred for 24 h at 25° C. The resulting mixture was then dialyzed (MWCO 3.5 kDa) against 0.1M ammoniumcarbonate, and the retentate was freeze-dried to obtain AFDye-488-click-Cellophil. The reaction was followed by GPC, and the structure of the obtained compound was verified by NMR spectroscopy. The fluorophore-labeled Cellophil derivative can be used to perform pharmacokinetic studies, e.g., to determine the half-life of the copolymer in the bloodstream or its renal elimination where a strong readout signal is advantageous.

### Example 42 Coupling of Cellophil Tetrazine-[DMA₃₀/AK-DOTA) to a TCO-Modified Protein

A solution of tetrazine-functionalized Cellophil copolymer of example 40 (3 eq.) is dissolved in PBS (pH 7.4) and a transcyclooctene (TCO)-modified protein [that might be prepared by a similar method as that presented in example 36 by substituting NH₂—PEG₅-TCO for NH₂-PEG₅-Azide] (1 eq. containing 2 TCO-groups) dissolved in PBS (pH 7.4) is added dropwise under stirring at room temperature for 3 h. Unreacted polymer is subsequently removed by dialysis using a membrane with a MWCO of 100 kDa. Success of the reaction is followed by SDS-PAGE and HPLC.

### Example 43 Synthesis of AK-DOTA

To a solution of AK (50 mg, 250 μmol) and Et₃N (104 μL, 749 μmol) in dry DMF (1 mL) was added DOTA-NHS (HPF₆/TFA salt) (200 mg, 262 μmol). The reaction mixture was stirred overnight at RT and filtered through a pad of cotton. The filtrate was precipitated in MeCN and then filtered. The cake was washed with MeCN and dried under reduced pressure to obtain a white powder (95 mg, 65%). This compound may be used for directed incorporation of chelating agents into the copolymer during polymerization.

1. A copolymer containing multiple molecules of an active agent made by
(a) polymerization of a reaction mixture comprising
(1) one or more polymerizable principal monomers, which monomers are characterized as having at least one vinylic group and not containing an amino acid residue,
(2) one or more co-principal monomers of formulae I or II in which at least one of Y and Z is H,
(3) optionally one or more co-principle monomers of any of formulae III to X
(4) an agent for controlling radical polymerization and
(5) an initiator system for generating free radical species, the polymerization yielding a copolymer;
(b) optionally, functionalization of the copolymer with a cell type-specific or a tissue type-specific targeting moiety; and
(c) coupling of the active agent to the copolymer after step (a) or optional step (b).

2. The copolymer containing multiple molecules of an active agent of claim 1, wherein the copolymer has an average molecular weight of 5,000 Daltons to 100,000 Daltons.

3. The copolymer containing multiple molecules of an active agent of claim 1, wherein at least 80% (w) of the copolymer molecules have an average molecular weight of 5,000 Daltons to 100,000 Daltons.

4. The copolymer containing multiple molecules of an active agent of claim 1, wherein the agent for controlling radical polymerization is a RAFT agent.

5. The copolymer containing multiple molecules of an active agent of claim 4, the copolymer made in two successive polymerization reactions,
wherein the first polymerization reaction is carried out in a first reaction mixture comprising one or more polymerizable principal monomers not containing an amino acid group, a RAFT agent for controlling the copolymerization, and an initiator system for generating free radical species, the polymerization yielding a RAFT pre-polymer, and
wherein the second polymerization reaction is carried out in a second reaction mixture comprising the RAFT pre-polymer of the first polymerization reaction, one or more co-principle monomers of formulae I and/or II, optionally one or more co-principal monomers of formula III-X, optionally one or more polymerizable principal monomers not containing an amino acid group, and an initiator system for generating free radical species.

6. The copolymer containing multiple molecules of an active agent of claim 4, wherein the RAFT agent contains a monodisperse spacer of 5-25 units.

7. The copolymer containing multiple molecules of an active agent of claim 4, wherein the RAFT agent contains a reactive group that is used for functionalization of the copolymer with a cell type-specific or a tissue type-specific targeting moiety.

8. The copolymer containing multiple molecules of an active agent of claim 7, wherein the reactive group is a thiol, an aldehyde, an alkyne, an azide, a tetrazine, a strained-alkene, an amine, a carboxyl, an ester, a diazirine, a phenyl azide, a thioester, a diazo, a Staudinger reactive phosphinoester (or phosphinothioester), a hydrazine, an oxime, an acylate to perform aza-Michael ligations or a motif capable of being used in an enzymatic coupling reaction.

9. The copolymer containing multiple molecules of an active agent of claim 8, wherein the motif is an oligo-glycine comprising 2-8 amino acid units and enabling sortase-mediated coupling reactions, a transglutaminase reactive substrate, an aldehyde tag or an autocatalytic intein sequence.

10. The copolymer containing multiple molecules of an active agent of claim 4, wherein the RAFT group of the RAFT agent is eliminated subsequent to copolymerization or functionalization of the copolymer.

11. A copolymer containing multiple molecules of an active agent made by polymerization of a reaction mixture comprising
(a) one or more polymerizable principal monomers, which monomers are characterized as having at least one vinylic group and not containing an amino acid residue,
(b) one or more co-principal monomers of formulae III to X,
(c) optionally one or more co-principal monomers of formula I and/or formula II,
(d) an agent for controlling radical polymerization, and
(e) optionally, functionalization of the copolymer with a cell type-specific or a tissue type-specific targeting moiety; and
(f) an initiator system for generating free radical species.

12. The copolymer containing multiple molecules of an active agent of claim 11, wherein the copolymer has an average molecular weight of 5,000 Daltons to 100,000 Daltons.

13. The copolymer containing multiple molecules of an active agent of claim 11, wherein the agent for controlling radical polymerization is a RAFT agent.

14. The copolymer containing multiple molecules of an active agent of claim 13, wherein the RAFT agent contains a reactive group that is used for functionalization of the copolymer with a cell type-specific or a tissue type-specific targeting moiety.

15. The copolymer containing multiple molecules of an active agent of claim 14, wherein the reactive group is a thiol, an aldehyde, an alkyne, an azide, a tetrazine, a strained-alkene, an amine, a carboxyl, an ester, a diazirine, a phenyl azide, a thioester, a diazo, a Staudinger reactive phosphinoester (or phosphinothioester), a hydrazine, an oxime, an acrylate to perform aza-Michael ligations or a motif capable of being used in an enzymatic coupling reaction.

16. The copolymer containing multiple molecules of an active agent of claim 15, wherein the motif is an oligo-glycine comprising 2-8 amino acid units and enabling sortase-mediated coupling reactions, a transglutaminase reactive substrate, an aldehyde tag or an autocatalytic intein sequence.

17. The copolymer containing multiple molecules of an active agent of claim 13, wherein the RAFT group of the RAFT agent is eliminated subsequent to copolymerization or functionalization of the copolymer.

18. A pharmaceutical composition comprising an effective amount of a copolymer containing multiple molecules of an active agent of claim 1 and a pharmaceutically acceptable carrier or excipient.

19. Use of the pharmaceutical composition of claim 18 for the treatment of a cancer or another disease or condition in a subject, comprising administering the pharmaceutical composition to the subject.
